SEQUENCE LISTING (41 sequences)

SEQ ID NO: 1 - protein, 238 aa, Artificial sequence
  synthetic sequence
MKTKKLLIATVTLATGLLGILPLTSMKLRVENPKKAQKHFVQNLNNVVFTNKELEDIYNLSNKEETKEVLKLFKLKVNQFYRHAFGIVNDYNGLLEYKEIFNMMFLKLSVVFDTQRKEANNVEQIKRNIAILDEIMAKADNDLSYFISQNKNFQELWDKAVKLTKEMKIKLKGQKLDLRDGEVAINKVRELFGSDKNVKELWWFRSLLVKGVYLIKRYYEGDIELKTTSDFAKAVFED

SEQ ID NO: 2 - protein, 138 aa, Artificial sequence
  synthetic sequence
  SITE (4)..(4): Xaa can be any naturally occurring amino acid
  SITE (7)..(7): Xaa can be any naturally occurring amino acid
  SITE (10)..(11): Xaa can be any naturally occurring amino acid
  SITE (14)..(15): Xaa can be any naturally occurring amino acid
  SITE (19)..(20): Xaa can be any naturally occurring amino acid
  SITE (22)..(22): Xaa can be any naturally occurring amino acid
  SITE (79)..(80): Xaa can be any naturally occurring amino acid
  SITE (83)..(83): Xaa can be any naturally occurring amino acid
  SITE (86)..(86): Xaa can be any naturally occurring amino acid
  SITE (90)..(91): Xaa can be any naturally occurring amino acid
  SITE (93)..(93): Xaa can be any naturally occurring amino acid
  SITE (115)..(115): Xaa can be any naturally occurring amino acid
EEEXLRXFKXXAEXXASGXXSXQVVFTNKELEDIYNLSNKEETKEVLKLFKLKVNQFYRHAFGIVNDYNGLKEYKYIFXXMFXLLXAVFXXQXKEANNVEQIKRNIAILDEIMAXADNDLSYMISQGSEQKLISEEDL

SEQ ID NO: 3 - protein, 124 aa, Artificial sequence
  synthetic sequence
  SITE (12)..(16): Xaa can be any naturally occurring amino acid
MKLRCENPKKAXXXXXQNLNNVVFTNKELEDIYDLSNKEETKEVLKLFKLKVNQFYRHAFGIVNDYNGLLEYKEIFNMMFLKLSVVFDTQRKEANNVEQIKRNIAILDEIMAKADNDLCYFISQ

SEQ ID NO: 4 - protein, 120 aa, Artificial sequence
  synthetic sequence
  SITE (12)..(19): Xaa can be any naturally occurring amino acid
MKLRCENPKKAXXXXXXXXNNVVFTNKELEDIYDLSNKEETKEVLKLFKLKVNQFYRHAFGIVNDYGDKEIFNMMFLKLSVVFDTQRKEANNVEQIKRNIAILDEIMAKADNDLCYFISQ

SEQ ID NO: 5 - protein, 120 aa, Artificial sequence
  synthetic sequence
  SITE (12)..(14): Xaa can be any naturally occurring amino acid
  SITE (16)..(16): Xaa can be any naturally occurring amino acid
  SITE (77)..(78): Xaa can be any naturally occurring amino acid
  SITE (81)..(81): Xaa can be any naturally occurring amino acid
  SITE (84)..(84): Xaa can be any naturally occurring amino acid
  SITE (87)..(89): Xaa can be any naturally occurring amino acid
  SITE (95)..(95): Xaa can be any naturally occurring amino acid
  SITE (98)..(98): Xaa can be any naturally occurring amino acid
  SITE (101)..(102): Xaa can be any naturally occurring amino acid
MKLRCENPKKAXXXNXQNLNNVVFTNKELEDIYDLSNKEETKEVLKLFKLKVNQFYRHAFGIVNDYGDKEIFNMMFXXLWXVFXSQXXXANNVEXIKXNIXXLDWIMAEADNDLCYFISQ

SEQ ID NO: 6 - protein, 120 aa, Artificial sequence
  synthetic sequence
  SITE (10)..(16): Xaa can be any naturally occurring amino acid
MKLRCENPKXXXXXXXQNLNNVVFTNKELEDIYDLSNKEETKEVLKLFKLKVNQFYRHAFGIVNDYGDKEIFNMMFMLLWRVFRSQRIDANNVELIKFNIRVLDWIMAEADNDLCYFISQ

SEQ ID NO: 7 - protein, 13 aa, Artificial sequence
  synthetic sequence
PKYVKQNTLKLAT

SEQ ID NO: 8 - protein, 15 aa, Artificial sequence
  synthetic sequence
LLEFYLAMPFATPME

SEQ ID NO: 9 - protein, 15 aa, Artificial sequence
  synthetic sequence
PVSKMRMATPLLMQA

SEQ ID NO: 10 - protein, 124 aa, Artificial sequence
  synthetic sequence
MKLRCENPKKAERHLLQNLNNVVFTNKELEDIYDLSNKEETKEVLKLFKLKVNQFYRHAFGIVNDYNGLLEYKEIFNMMFLKLSVVFDTQRKEANNVEQIKRNIAILDEIMAKADNDLCYFISQ

SEQ ID NO: 11 - protein, 124 aa, Artificial sequence
  synthetic sequence
MKLRCENPKKAYVLGVQNLNNVVFTNKELEDIYDLSNKEETKEVLKLFKLKVNQFYRHAFGIVNDYNGLLEYKEIFNMMFLKLSVVFDTQRKEANNVEQIKRNIAILDEIMAKADNDLCYFISQ

SEQ ID NO: 12 - protein, 124 aa, Artificial sequence
  synthetic sequence
MKLRCENPKKAVRWGGQNLNNVVFTNKELEDIYDLSNKEETKEVLKLFKLKVNQFYRHAFGIVNDYNGLLEYKEIFNMMFLKLSVVFDTQRKEANNVEQIKRNIAILDEIMAKADNDLCYFISQ

SEQ ID NO: 13 - protein, 124 aa, Artificial sequence
  synthetic sequence
MKLRCENPKKAQLFLAQNLNNVVFTNKELEDIYDLSNKEETKEVLKLFKLKVNQFYRHAFGIVNDYNGLLEYKEIFNMMFLKLSVVFDTQRKEANNVEQIKRNIAILDEIMAKADNDLCYFISQ

SEQ ID NO: 14 - DNA, 360 bp, Artificial sequence
  synthetic sequence
  misc_feature (34)..(35): n is a, c, g, or t
  misc_feature (37)..(38): n is a, c, g, or t
  misc_feature (40)..(41): n is a, c, g, or t
  misc_feature (242)..(242): n is a, c, g, or t
  misc_feature (250)..(250): n is a, c, g, or t
atgaagctgcgttgtgaaaatccgaagaaagccnnknnknnkaacsyacaaaacctaaataacgtcgtcttcaccaacaaagagcttgaagacatctatgatctgtcaaataaggaagagacaaaggaagtccttaagctgttcaaacttaaagtgaaccagttctatagacatgctttcggtattgtcaatgactatggagataaggagatctttaacatgatgttcvhsmtgctatggvnsgtgttcnkcagtcagarahdcrvcgccaacaacgtagagstaattaaattmaacatcaravtgttagattggataatggcggaagcagacaatgacttatgttacttcattagtcaa

SEQ ID NO: 15 - protein, 9 aa, Artificial sequence
  synthetic sequence
SLLMWITQV

SEQ ID NO: 16 - protein, 120 aa, Artificial sequence
  synthetic sequence
MKLRCENPKKAYRDNAQNLNNVVFTNKELEDIYDLSNKEETKEVLKLFKLKVNQFYRHAFGIVNDYGDKEIFNMMFMLLWRVFRSQRIDANNVELIKFNIRVLDWIMAEADNDLCYFISQ

SEQ ID NO: 17 - protein, 5 aa, Artificial sequence
  synthetic sequence
EVHMV

SEQ ID NO: 18 - protein, 5 aa, Artificial sequence
  synthetic sequence
DNHLV

SEQ ID NO: 19 - protein, 5 aa, Artificial sequence
  synthetic sequence
ETHWV

SEQ ID NO: 20 - protein, 5 aa, Artificial sequence
  synthetic sequence
ESHLV

SEQ ID NO: 21 - protein, 5 aa, Artificial sequence
  synthetic sequence
ERHLL

SEQ ID NO: 22 - protein, 5 aa, Artificial sequence
  synthetic sequence
DNHLV

SEQ ID NO: 23 - protein, 5 aa, Artificial sequence
  synthetic sequence
EVHWV

SEQ ID NO: 24 - protein, 5 aa, Artificial sequence
  synthetic sequence
DWHLV

SEQ ID NO: 25 - protein, 5 aa, Artificial sequence
  synthetic sequence
FRWGG

SEQ ID NO: 26 - protein, 5 aa, Artificial sequence
  synthetic sequence
VRWGG

SEQ ID NO: 27 - protein, 5 aa, Artificial sequence
  synthetic sequence
VRWGG

SEQ ID NO: 28 - protein, 5 aa, Artificial sequence
  synthetic sequence
VKWGG

SEQ ID NO: 29 - protein, 5 aa, Artificial sequence
  synthetic sequence
YVLGV

SEQ ID NO: 30 - protein, 5 aa, Artificial sequence
  synthetic sequence
GRFGV

SEQ ID NO: 31 - protein, 5 aa, Artificial sequence
  synthetic sequence
YRWGG

SEQ ID NO: 32 - protein, 5 aa, Artificial sequence
  synthetic sequence
VRWGG

SEQ ID NO: 33 - protein, 5 aa, Artificial sequence
  synthetic sequence
STYLA

SEQ ID NO: 34 - protein, 5 aa, Artificial sequence
  synthetic sequence
SIFLA

SEQ ID NO: 35 - protein, 5 aa, Artificial sequence
  synthetic sequence
QLFLA

SEQ ID NO: 36 - protein, 5 aa, Artificial sequence
  synthetic sequence
VVFLV

SEQ ID NO: 37 - protein, 5 aa, Artificial sequence
  synthetic sequence
VLFLV

SEQ ID NO: 38 - protein, 5 aa, Artificial sequence
  synthetic sequence
AVYLA

SEQ ID NO: 39 - protein, 5 aa, Artificial sequence
  synthetic sequence
AVYGA

SEQ ID NO: 40 - protein, 5 aa, Artificial sequence
  synthetic sequence
TAYGA

SEQ ID NO: 41 - protein, 213 aa, Artificial sequence
  synthetic sequence
MKLRVENPKKAQKHFVQNLNNVVFTNKELEDIYDLSNKEETKEVLKLFKLKVNQFYRHAFGIVNDYNGLLEYKEIFNMMFLKLSVVFDTQRKEANNVEQIKRNIAILDEIMAKADNDLSYFISQNKNFQELWDKAVKLTKEMKIKLKGQKLDLRDGEVAINKVRELFGSDKNVKESWWFRSLLVKGVYLIKRYYEGDIELKTTSDFAKAVFED

(19) United States
(12) Patent Application Publication
(10) Pub. No.: US 2023/0265195 A1
(43) Pub. Date: Aug. 24, 2023

(54) TARGET-RECOGNITION OF ANTIGEN-MHC COMPLEX REPORTER (TRACER) PLATFORM

(71) Applicant: The Board of Trustees of the Leland Stanford Junior University, Stanford, CA (US)

(72) Inventors: Possu Huang, Stanford, CA (US); Haotian Du, Redwood City, CA (US)

(21) Appl. No.: 18/016,243

(22) PCT Filed: Jul. 30, 2021
(86) PCT No.: PCT/US2021/044069

§ 371 Date: Jan. 13, 2023

Related U.S. Application Data
(60) Provisional application No. 63/059,767, filed on Jul. 31, 2020.

(51) Int. Cl. C07K 16/28

(52) U.S. Cl. CPC C07K 16/2833; C07K 16/2809; C07K 2317/24; C07K 2317/52; C07K 2317/92

(57) ABSTRACT

Abstract: Compositions and methods are provided relating to the design, screening and therapeutic use of designed binding proteins that specifically interact with an MHC/peptide complex. Proteins that specifically bind to an MHC/antigenic peptide complex of interest are designed through engineering of protein structure and screening assays. Selected binders are screened to minimize cross-reactivity with self-MHC-peptides. The antigen binding region thus developed can be formatted into a therapeutic agent that activates cytolytic pathways.

### CROSS REFERENCE TO RELATED APPLICATION

This application claims the benefit of PCT Application No. PCT/US2021/044069, filed Jul. 30, 2021, which claims priority to U.S. Provisional Patent Application No. 63/059,767 filed Jul. 31, 2020,-which applications are incorporated herein by reference in their entirety.

### INCORPORATION BY REFERENCE OF SEQUENCE LISTING

A Sequence Listing is provided herewith as a Sequence Listing text, STAN-1775WO_SEQ_LIST_ST25 created on Jan. 13, 2023 and having a size of 24,156 bytes. The contents of the Sequence Listing text are incorporated herein by reference in their entirety.

### BACKGROUND

In a number of diseases, such as cancer, viral infection, autoimmunity, etc., the expression of proteins by a cell results in the presentation of antigenic peptides on host cells’ major histocompatibility complex (MHC) molecules, particularly MHC Class I proteins. The nature of the disease determines which peptides stimulate an immune response, e.g. autoantigens in autoimmune disease; pathogen peptides during infection, neoantigens in cancer. The MHCs, with their loaded cognate peptides, act essentially as cellular barcodes that report on cellular health. While peptides from most proteins synthesized by a cell are presented on the cell surface, T-cell receptors interact specifically with MHC to identify the presence of foreign antigens among the self-peptides.

It is highly desirable to use the peptide-loaded MHCs (pMHCs) for identifying and targeting diseased cells, such as virus-infected cells, cancer cells, etc. However, the engineering of antigen-specific T cell receptors (TCRs) for this purpose remains a challenge. Leading technologies for engineering TCRs use mutagenic libraries or TCRs′ natural genetic repertoire to discover TCRs that respond to specific pMHCs. But due to the unique docking geometry required, as well as central tolerance imposing restrictions on TCR affinity, engineered TCRs have been shown to either be sub-optimal in binding or exhibit unexpected cross-reactivity.

A widely applicable technology to identify and report on the status of the cells, similar to immunosurveillance, is lacking. If specific transformed cell pMHC targeting is achievable, such a technology would be transformative for our ability to detect and control the spread of viruses and cancer inside the body. The present disclosure addresses this need.

### SUMMARY

Compositions and methods are provided relating to the design, screening and therapeutic use of de novo designed binding proteins, with antigen binding regions, that specifically interact with MHC/peptide complexes. A benefit of specificity of a binding protein for an MHC/peptide antigen is the ability to recognize and interact with polypeptide sequences that are normally intracellular, unless processed and presented by MHC proteins. Proteins that specifically bind to an MHC/antigenic peptide complex of interest are designed through engineering protein structure, targeted mutagenesis, and screening assays. The term TRACeR may be referred to herein as a designed protein that provides for binding to an antigenic peptide in the context of the MHC presentation. In addition to designing and screening for peptide antigen specificity, the MHC allelic specificity and affinity of the TRACeR can also provide a basis for protein engineering by amino acid modifications and screening. The antigen binding region thus developed may be formatted into a therapeutic agent that activates cytolytic pathways, e.g. by fusion to an Fc effector region, as a CAR-T construct, as a CD3-based bispecific T-cell engager, and the like. For autoimmune indications the antigen binding region may be formatted to block interactions without activating immune cytotoxic pathways.

In some embodiments, polypeptide libraries and methods are provided for generating and identifying designed binding proteins that have affinity for an MHC/peptide complex of interest. In some such methods, a library of designed TRACeR binding proteins is generated, where the polypeptides in the library comprise an engineered peptide recognition element (PRE) sequence and MHC interacting surfaces. The scaffold of the TRACeR binding protein localizes antigen recognition to a single structural element, and provides specificity and affinity in combination with an engineered PRE loop. A PRE loop, as disclosed herein, resides within the first 20 amino acids of a TRACeR sequence, or at an equivalent position as aligned to reference SEQ ID NO:1. A PRE loop sequence is randomized at one or more residues in libraries, according to the sequence formulas provided herein.

In some embodiments, the TRACeR scaffold comprises a polypeptide having a sequence as disclosed herein, and libraries of such polypeptides including, for example, any of SEQ ID NO:2, 3, 4, 5, or 6, where “randomized”, or variable residues are as indicated. Compositions of interest include polypeptide libraries; polynucleotide libraries encoding the polypeptides, cells expressing such polynucleotide libraries, and individual polynucleotides, cells and polypeptides isolated therefrom, included polypeptides formatted for therapeutic efficacy, e.g. CAR-T, Fc fusion, etc.

In some embodiments a TRACeR library is provided for screening MHC Class II-peptide complexes. In some embodiments the TRACeR library has a sequence according to the formula set forth in any of SEQ ID NO:2, SEQ ID NO:3 or SEQ ID NO:4. Degeneracy is generated at the variable residues indicated in the sequence formulas to create a library of protein sequences, which library can be screened for specific peptides presented in the Class II HLA context. In some embodiments, for example and without limitation, SEQ ID NO:3, residues 5 and 119; SEQ ID NO:4, residues 5 and 115, the TRACeR polypeptides comprise cysteine residues that form a stabilizing disulfide bond. Individual TRACeR proteins with specificity for an MHC/peptide complex of interest are identified by screening, and can be further used in a format that provides for therapeutic efficacy. SEQ ID NO:4 is of particular interest for this purpose. In some such embodiments the HLA class II protein is human HLA-DR1.

In some embodiments a TRACeR library is provided for screening MHC Class I-peptide complexes. In some embodiments the TRACeR library has a sequence according to the formula set forth in any of SEQ ID NO:5 or SEQ ID NO:6. Degeneracy is generated at the variable residues indicated in the sequence formulas to create a library of protein sequences, which library can be screened for specific peptides presented in the Class I HLA context. SEQ ID NO:5 provides a scaffold for screening TRACeR polypeptides that bind to an HLA Class I allele of interest. SEQ ID NO:6 provides a sequence formula for a library useful in screening MHC Class I/peptide complexes. In some embodiments, for example and without limitation, SEQ ID NO:5, residues 5 and 115; SEQ ID NO:6, residues 5 and 115, the TRACeR polypeptides comprise cysteine residues that form a stabilizing disulfide bond. Individual TRACeR proteins with specificity for an MHC-peptide combination of interest are identified by screening, and can be further used in a format that provides for therapeutic efficacy. SEQ ID NO:6 is of particular interest for this purpose. In some such embodiments the MHC protein is human HLA-A*02.

A TRACeR library is initially generated as a population of polynucleotides encoding binding proteins, e.g. according to the sequence formula of any of SEQ ID NO:2-6, operably linked to an expression vector, which library may comprise at least 10⁶, at least 10⁷, more usually at least 10⁸ different coding sequences, and may contain up to about 10¹³, 10¹⁴ or more different sequences. The library is introduced into a suitable host cell that expresses the encoded polypeptide, which host cells include, without limitation, yeast cells. The number of unique host cells expressing the polypeptide is generally less than the total predicted diversity of polynucleotides, e.g. up to about 10⁹ specificities. For example, a fusion protein including the designed binding protein can be secreted to the extracellular space and anchored to the cell wall of a yeast cell. As a result, the protein of interest is displayed on the cell surface where it is accessible by soluble ligands. The library of cells expressing the designed binding protein(s) is then contacted with an MHC-peptide complex of interest. A labeled multimeric MHC I or MHC II protein bound to a peptide antigen of interest is contacted with the cell library, and sorted according to that label for cells that bind to that MHC/peptide complex. The labeled MHC/peptide complex may be a soluble multimer, e.g. a tetramer, a dendrimer, etc. Screening may be performed in multiple rounds to select for binding that is high affinity, pan-allelic, etc.

Various peptides are useful for TRACeR binding when complexed to an MHC, for example and without limitation, pathogen peptides including viral, bacterial, protozoan, etc. sequences, known or predicted cancer antigens, autoantigens, and the like. Viral peptides can be utilized from any virus of interest, including without limitation RNA viruses, such as coronavirus, e.g. SARS-CoV1, SARS-CoV2, MERS-CoV, etc. Cancer antigens, e.g. neoantigens, include sequences that may be over-expressed, expressed in non-normal tissues, comprise antigenic mutations, and the like. (see for example Schumacher et al. (2019) Annu Rev Immunol 37:173-200); or predicted from databases such as The Cancer Genome Atlas database. Autoantigens include, for example, antigens expressed in MS, IDDM, RA, etc. Other peptides can be generated de novo, or predicted from sequences of interest.

MHC proteins of interest include any of the mammalian MHC proteins. Human HLA proteins are of interest, including HLA Class I proteins, e.g. human HLA-A, HLA-B, HLA-C. In some embodiments an allele of HLA-A or HLA-B is screened. As will be appreciated by one with skill in the art, the HLA locus is highly polymorphic and a large number of sequence variants are known and described in the art, including without limitation any of the HLA-A*01, HLA-A*02, up to HLA-A*80 alleles and serotypes thereof; and the HLA-B*07, HLA-B*08 up to HLA-B*83 and serotypes thereof. MHC sequences used for screening purposes typically comprise the peptide binding region, e.g. the alpha 1 and alpha 2 domains, or the portion of those domains required to form a peptide binding complex. The MHC sequences for screening may be a naturally occurring sequence, or may be a designed consensus sequence to provide pan-allelic specificity. HLA Class II proteins are also of interest, e.g. HLA-DPA1, HLA-DPB1, HLA-DQA1, HLA-DQB1, HLA-DR1, HLA-DRA, HLA-DRB1, etc.

Selected binders can be screened to minimize cross-reactivity with non-cognate MHC/peptide complexes, e.g. by pair-wise screening against non-cognate peptides, including without limitation self-peptides, usually against peptides presented in the same MHC context as the cognate peptide. By contrasting the signals from the positive and negative controls, constructs that have the potential to cross-react with self-peptides are identified and eliminated. Further screening of candidate binders may utilize, for example, x-scan, where the peptide antigen is subjected to saturation mutagenesis and screened against known antigens bioinformatically, and if required, against proteins or cell lines.

In an embodiment, an individual TRACeR sequence that provides for specific binding to an antigenic peptide in an MHC context, as described above, is covalently linked, e.g. conjugated or fused, to an effector polypeptide, e.g. an immunoglobulin effector sequence, for example an Fc sequence, etc., in a chimeric antigen (CAR), in a CD3-based bispecific T-cell engager, and the like to generate a therapeutic entity. A TRACeR polypeptide or therapeutic entity derived therefrom may be labeled with a detectable label, immobilized on a solid phase and/or conjugated with a heterologous compound, e.g. toxin, etc.

In some embodiments, an engineered cell is provided, e.g. an engineered T cell, engineered B cell, etc., in which the cell has been modified by introduction of a TRACeR coding sequence, which is optionally formatted as a chimeric antigen receptor (CAR), immunoglobulin effector protein, CD3-based bispecific T-cell engager, etc. The engineered cell can be provided in a unit dose for therapy, and can be allogeneic, autologous, etc., with respect to an intended recipient. Introduction of the coding sequence can be performed in vivo or in vitro, using any appropriate vector, e.g., viral vectors, integrating vectors, and the like.

In some embodiments, a therapeutic method is provided, particularly relating to the elimination of virus infected cells or cancer cells, by administering to an individual an effective dose of a therapeutic entity or cell expressing a therapeutic entity as described above.

In some embodiments, a vector comprising a polynucleotide sequence encoding a polypeptide comprising all or part of a TRACeR sequence or fusion protein comprising a TRACeR sequence is provided, where the coding sequence is operably linked to a promoter active in the desired cell. In some embodiments, the promoter may be constitutive or inducible. Various vectors are known in the art and can be used for this purpose, e.g. viral vectors, plasmid vectors, minicircle vectors, etc. which vectors can be integrated into the target cell genome, or can be episomally maintained. The vector may be provided in a kit.

In some embodiments, a kit is provided for the identification of protein sequences that bind to an MHC-peptide complex of interest. Such a kit may comprise a library of polynucleotides encoding a TRACeR sequence, e.g. of SEQ ID NO:2, 3, 4, 5, 6, etc., where a diverse set of sequences is provided, e.g. at least 10⁶, at least 10⁷, more usually at least 10⁸, at least 10⁹, at least 10¹⁰ different sequences are present in the library and may contain up to about 10¹⁴ different ligands, usually up to about 10¹³ different ligands. The polynucleotide library can be provided as a population of transfected cells, or as an isolated population of nucleic acids. Reagents for labeling and multimerizing an MHC-peptide complex can be included. In some embodiments the kit will further comprise a software package for analysis of a sequence database.

### BRIEF DESCRIPTION OF THE DRAWINGS

FIGS. 1A-1H provide binding plots and structures for engineered binding proteins with different, incremental modifications. The binding compares specificity for both HA and CLIP peptide. The native protein is shown in A. Native MAM is a two-domain protein, with one MHC binding domain on N terminal and one TCR binding domain on C terminal. In B and C truncated versions are tested, showing the truncated N terminal domain of MAM is able to fold properly by itself and maintain MHC binding. D illustrates the truncated structure. Shown in panel E. is a truncated protein with an engineered disulfide bond. The N terminal loop is very long and flexible in structure, and introducing a disulfide bond to restrict loop conformation could help with stability and binding. The disulfide bond holds an end of this flexible loop, allowing introduction of a PRE region into the loop. A structure is shown in F. Panel G shows the protein with an edited alpha alpha turn connection into a more energy favored geometry, with helix well-capped, however binding affinity was then weaker, illustrated in H.

FIG. 2 is a schematic for using high through-put screening to evolve peptide-specific MHC binders. Starting from the redesigned MAM protein, (SEQ ID NO:3) 5 residues on the loop that interact with peptide were made variable with randomized codon NNK, which include all 20 amino acids in one position. The library is screened with FACS for constructs that bind specifically with target peptides. The diversity for the first library is about 3.2×10⁶. Specific binders from the same library for three targets, HA, NYESO-1, and CLIP on the same HLA-DR1 allele were screened. A first screen selects for positive binding for target, then the selected binders are screened for negative binding to non-cognate peptides.

FIG. 3 is an example of screening results. Round 1 is gated for positive binding signal on target. Round 2 is gated for affinity and specificity using cognate target tetramer as positive selection and the non-cognate as negative selection.

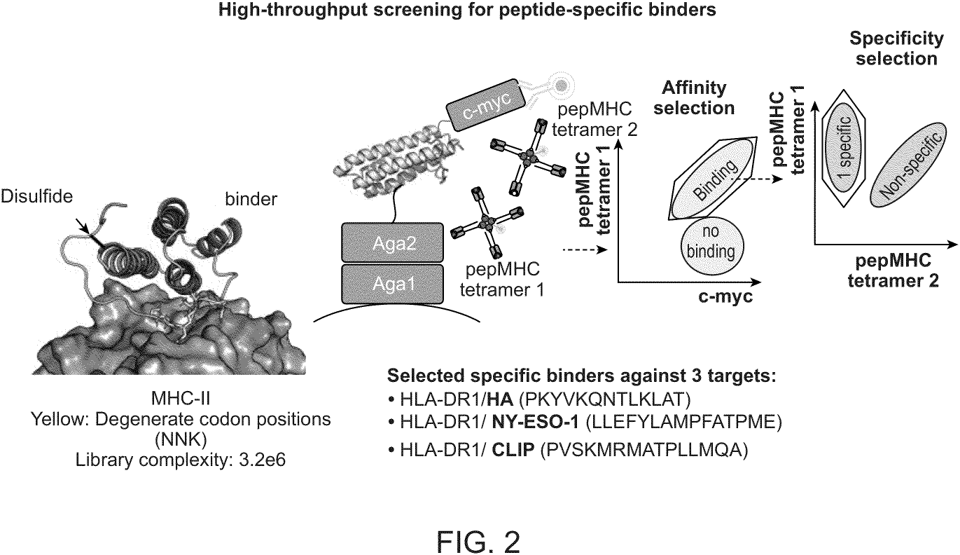

FIG. 4 is an example showing the specificity of single clones, transformed back into yeast.

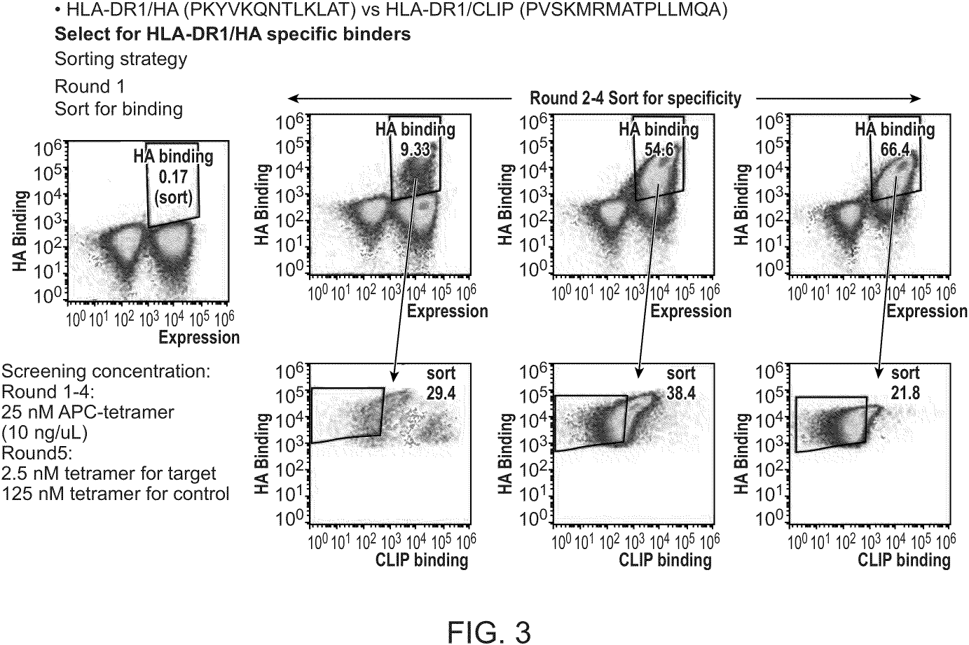

FIG. 5 is a schematic of the process for designing MHC Class I binding proteins. Patchdock and rifdock were used to find initial docking poses between MHC-II binders and MHC-I targets, which were iteratively refined the docking poses with rosettadock and design the interface sequence with fast design and fast relax movers in rosettascripts to identify diverse sequence mutations on the interface that are compatible with a given docking pose. After running thousands of parallel jobs, the model successfully converged to a low energy docking funnel with a docking mode similar with how MAM binds MHC-II.

FIG. 6. A subset of mutations suggested by computational modeling were generated and utilized into yeast combinatorial libraries. Sequence diversity are mainly from two regions, one is the loop, the other is the helical bundle interface that touches MHC groove. Swiftlib was used to design randomized codons that can cover diverse amino acid positions. The final designed library size is ~1.3×10¹¹, although the first library complexity was ~1×10⁹. The first target was NY-ESO-1 peptide presented by HLA-A*02 allele.

FIG. 7 is an example of screening to select a binding population with the HLA-A2 NYESO-1 target.

FIG. 8 Screening assay for HLA allele specificity (A) using the MHC-II tetramer reagent and MHC-I tetramer reagent with another allele as controls; and peptide specificity (B) using the same HLA-A2 allele with different peptides as negative controls.

FIG. 9 Designed peptide-specific MHC binders can be implemented as Fc fusions for recruiting NK cells or macrophages.

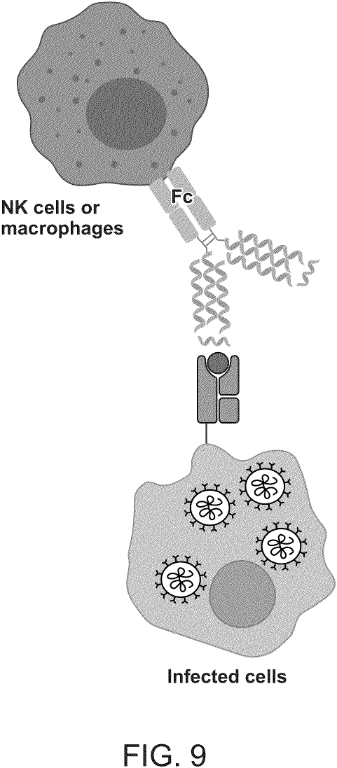

FIG. 10 provides an overview of a construct for yeast expression and screening protocol. Shown in (A), is the construct, where a synthetic library sequence based on the MAM protein is indicated as “protein”, which is flanked by two epitope tags: a 9-amino acid hemagglutinin antigen (HA) tag and a 10-amino acid c-myc tag, and is fused to the C-terminus of the a-agglutinin Aga2p subunit. (B) Protein display on the yeast cell surface. Following translation, the 69-amino acid Aga2p subunit associates with 725-amino acid a-agglutinin Aga1p subunit via two disulfide bonds. The fusion protein is subsequently secreted to the extracellular space where Aga1p is anchored to the cell wall via a β1,6-glucan covalent linkage. As a result, the protein of interest is displayed on the cell surface where it is accessible by soluble ligands. A labeled multimeric MHC I or MHC II protein bound to a peptide antigen of interest is contacted with the yeast cells, and sorted according to the label for cells that bind to the MHC/peptide complex.

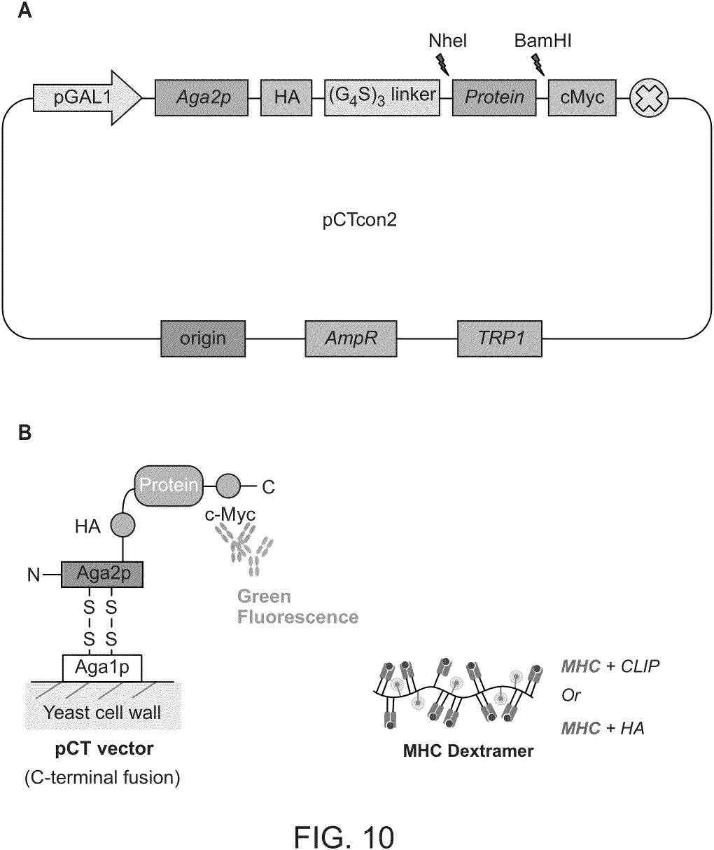

FIG. 11 provides certain relevant protein sequences. SEQ ID NO:1 is native MAM protein sequence. Synthetic library sequences are provided as SEQ ID NO:2; 3; 4; 5 and 6. The native MAM protein is truncated at the termini. Exemplified in SEQ ID NO:2, in a library sequence, residues 1-46 are a de novo designed peptide recognition element, a helix that replaces a loop present in MAM. The library sequence includes randomized positions for screening shown as *; and positions shown as # are randomized residues for the second step of the screening process.

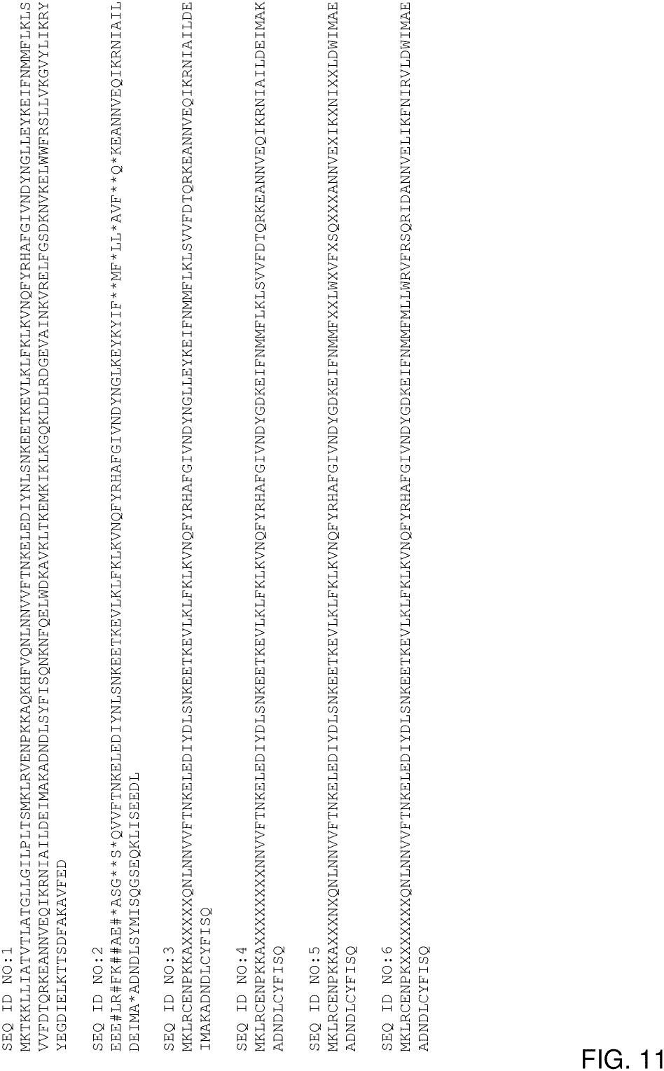

### DETAILED DESCRIPTION OF THE EMBODIMENTS

Before the subject invention is described further, it is to be understood that the invention is not limited to the particular embodiments of the invention described below, as variations of the particular embodiments may be made and still fall within the scope of the appended claims. It is also to be understood that the terminology employed is for the purpose of describing particular embodiments, and is not intended to be limiting. In this specification and the appended claims, the singular forms “a,” “an” and “the” include plural reference unless the context clearly dictates otherwise.

Where a range of values is provided, it is understood that each intervening value, to the tenth of the unit of the lower limit unless the context clearly dictates otherwise, between the upper and lower limit of that range, and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood to one of ordinary skill in the art to which this invention belongs. Although any methods, devices and materials similar or equivalent to those described herein can be used in the practice or testing of the invention, illustrative methods, devices and materials are now described.

All publications mentioned herein are incorporated herein by reference for the purpose of describing and disclosing the subject components of the invention that are described in the publications, which components might be used in connection with the presently described invention.

The present invention has been described in terms of particular embodiments that comprise preferred modes for the practice of the invention. It will be appreciated by those of skill in the art that, in light of the present disclosure, numerous modifications and changes can be made in the particular embodiments exemplified without departing from the intended scope of the invention. For example, due to codon redundancy, changes can be made in the underlying DNA sequence without affecting the protein sequence. Moreover, due to biological functional equivalency considerations, changes can be made in protein structure without affecting the biological action in kind or amount. All such modifications are intended to be included within the scope of the appended claims.

MHC Proteins. Major histocompatibility complex proteins (also called human leukocyte antigens, HLA, or the H2 locus in the mouse) are protein molecules expressed on the surface of cells that confer a unique antigenic identity to these cells. MHC/HLA antigens are target molecules that are recognized by T-cells and natural killer (NK) cells as being derived from the same source of hematopoietic reconstituting stem cells as the immune effector cells (“self”) or as being derived from another source of hematopoietic reconstituting cells (“non-self”). Two main classes of HLA antigens are recognized: HLA class I and HLA class II.

The MHC proteins used in the methods of the invention may be from any mammalian or avian species, e.g. primate sp., particularly humans; rodents, including mice, rats and hamsters; rabbits; equines, bovines, canines, felines; etc. Of particular interest are the human HLA proteins. Included in the HLA proteins are the class II subunits HLA-DPα, HLA-DPβ, HLA-DQα, HLA-DQβ, HLA-DRα and HLA-DRβ, and the class I proteins HLA-A, HLA-B, HLA-C, and β₂-microglobulin.

The MHC binding domains are typically a soluble form of the normally membrane-bound protein. The soluble form is derived from the native form by deletion of the transmembrane domain. Conveniently, the protein is truncated, removing both the cytoplasmic and transmembrane domains. In some embodiments, the binding domains of a major histocompatibility complex protein are soluble domains of Class II alpha and beta chain. In some such embodiments the binding domains have been subjected to mutagenesis and selected for amino acid changes that enhance the solubility of the single chain polypeptide, without altering the peptide binding contacts.

An “allele” is one of the different nucleic acid sequences of a gene at a particular locus on a chromosome. One or more genetic differences can constitute an allele. An important aspect of the HLA gene system is its polymorphism. Each gene, MHC class I (A, B and C) and MHC class II (DP, DQ and DR) exists in different alleles. Current nomenclature for HLA alleles is designated by numbers, as described by Marsh et al.: Nomenclature for factors of the HLA system, 2010. Tissue Antigens 75:291-455, herein specifically incorporated by reference. For HLA protein and nucleic acid sequences, see Robinson et al. (2011), The IMGT/HLA database. Nucleic Acids Research 39 Suppl 1:D1171-6, herein specifically incorporated by reference.

MHC context. The function of MHC molecules is to bind peptide fragments derived from pathogens or aberrant proteins derived from transformed cells, and display them on the cell surface for recognition by the appropriate T cells. Thus, T cell receptor recognition can be influenced by the MHC protein that is presenting the antigen. The term MHC context refers to the recognition by a TCR of a given peptide, when it is presented by a specific MHC protein.

Class II HLA/MHC. In some embodiments, the binding domains of a major histocompatibility complex protein are soluble domains of Class II alpha and beta chain. In some such embodiments the binding domains have been subjected to mutagenesis and selected for amino acid changes that enhance the solubility of the single chain polypeptide, without altering the peptide binding contacts.

Class I HLA/MHC. For class I proteins, the binding domains may include the a1, α2 and optionally α3 domain of a Class I allele, including without limitation HLA-A, HLA-B, HLA-C, H-2 K, H-2D, H-2L, which are combined with β₂-microglobulin. In certain specific embodiments, the binding domains are HLA-A2 binding domains, e.g. comprising at least the alpha 1 and alpha 2 domains of an A2 protein. A large number of alleles have been identified in HLA-A2, including without limitation HLA-A*02:01:01:01 to HLA-A*02:478, which sequences are available at, for example, Robinson et al. (2011), The IMGT/HLA database. Nucleic Acids Research 39 Suppl 1:D1171-6. Among the HLA-A2 allelic variants, HLA-A*02:01 is the most prevalent.

Peptide ligands are peptide antigens against which an immune response involving T lymphocyte antigen specific response can be generated. Such antigens include antigens associated with autoimmune disease, infection, cancer neoantigens, foodstuffs such as gluten, etc., allergy or tissue transplant rejection. Antigens also include various microbial antigens, e.g. as found in infection, in vaccination, etc., including but not limited to antigens derived from virus, bacteria, fungi, protozoans, parasites and tumor cells. Tumor antigens include tumor specific antigens, e.g. immunoglobulin idiotypes and T cell antigen receptors; oncogenes, such as p21/ras, p53, p210/bcr-abl fusion product; etc.; developmental antigens, e.g. MART-1/Melan A; MAGE-1, MAGE-3; GAGE family; telomerase; etc.; viral antigens, e.g. human papilloma virus, Epstein Barr virus, etc.; tissue specific self-antigens, e.g. tyrosinase; gp100; prostatic acid phosphatase, prostate specific antigen, prostate specific membrane antigen; thyroglobulin, α-fetoprotein; etc.; and self-antigens, e.g. her-2/neu; carcinoembryonic antigen, muc-1, and the like.

Library. In some embodiments of the invention, a library is provided of polypeptides, or of nucleic acids encoding such polypeptides, usually a library of different ligands, where at least 10⁶, at least 10⁷, more usually at least 10⁸ different peptide ligands are present in the library.

Conventional methods of assembling the coding sequences can be used. In order to generate the diversity of peptide ligands, randomization, error prone PCR, mutagenic primers, and the like as known in the art, are used to create a set of polynucleotides. The library of polynucleotides is typically ligated to a vector suitable for the host cell of interest. In various embodiments the library is provided as a purified polynucleotide composition encoding polypeptides, where the population of cells can be, without limitation yeast cells, and where the yeast cells may be induced to express the polypeptide library.

“Suitable conditions” shall have a meaning dependent on the context in which this term is used. That is, when used in connection with binding of a T cell receptor to a polypeptide of the formula polynucleotide composition encoding the P-L₁-β-L₂-α-L₃-T polypeptide, the term shall mean conditions that permit a TCR to bind to a cognate peptide ligand. When this term is used in connection with nucleic acid hybridization, the term shall mean conditions that permit a nucleic acid of at least 15 nucleotides in length to hybridize to a nucleic acid having a sequence complementary thereto. When used in connection with contacting an agent to a cell, this term shall mean conditions that permit an agent capable of doing so to enter a cell and perform its intended function. In one embodiment, the term “suitable conditions” as used herein means physiological conditions.

Sequencing platforms that can be used in the present disclosure include but are not limited to: pyrosequencing, sequencing-by-synthesis, single-molecule sequencing, second-generation sequencing, nanopore sequencing, sequencing by ligation, or sequencing by hybridization. Preferred sequencing platforms are those commercially available from Illumina (RNA-Seq) and Helicos (Digital Gene Expression or “DGE”). “Next generation” sequencing methods include, but are not limited to those commercialized by: 1) 454/Roche Lifesciences including but not limited to the methods and apparatus described in Margulies et al., Nature (2005) 437:376-380 (2005); and U.S. Pat. Nos. 7,244,559; 7,335,762; 7,211,390; 7,244,567; 7,264,929; 7,323,305; 2) Helicos BioSciences Corporation (Cambridge, MA) as described in U.S. application Ser. No. 11/167046, and U.S. Pat. Nos. 7501245; 7491498; 7,276,720; and in U.S. Pat. Application Publication Nos. US20090061439; US20080087826; US20060286566; US20060024711; US20060024678; US20080213770; and US20080103058; 3) Applied Biosystems (e.g. SOLiD sequencing); 4) Dover Systems (e.g., Polonator G.007 sequencing); 5) Illumina as described US Pat. Nos. 5,750,341; 6,306,597; and 5,969,119; and 6) Pacific Biosciences as described in US Pat. Nos. 7,462,452; 7,476,504; 7,405,281; 7,170,050; 7,462,468; 7,476,503; 7,315,019; 7,302,146; 7,313,308; and US Application Publication Nos. US20090029385; US20090068655; US20090024331; and US20080206764. All references are herein incorporated by reference. Such methods and apparatuses are provided here by way of example and are not intended to be limiting.

TRACeR. As used herein, the term TRACeR refers to a designed polypeptide that specifically binds to an MHC/peptide complex with a high affinity and specificity, e.g. with an affinity of less than about 10⁻⁶ M, less than about 10⁻⁷ M, less than about 10⁻⁸ M, less than about 10⁻⁹ M, or less. Examples of TRACeR sequence formulas for library generation are provided, for example and without limitation, by SEQ ID NO:2, 3, 4, 5 and 6.

The terms “specific binding,” “specifically binds,” and the like, refer to non-covalent or covalent preferential binding to a molecule relative to other molecules or moieties in a solution or reaction. In some embodiments, the affinity of one molecule for another molecule to which it specifically binds is characterized by a KD (dissociation constant) of 10⁻⁵ M or less (e.g., 10⁻⁶ M or less, 10⁻⁷ M or less, 10⁻⁸ M or less, 10⁻⁹ M or less, 10⁻¹⁰ M or less, 10⁻¹¹ M or less, 10⁻¹² M or less). “Affinity” refers to the strength of binding, increased binding affinity being correlated with a lower KD. In an embodiment, affinity is determined by surface plasmon resonance (SPR), e.g. as used by Biacore systems. The affinity of one molecule for another molecule is determined by measuring the binding kinetics of the interaction, e.g. at 25° C.

Chimeric antigen receptor (CAR). A CAR is comprised of the general structure where an antigen binding domain, e.g. a TRACeR sequence disclosed herein, usually provided in an scFv format, is linked to T cell receptor effector functions. The term refers to artificial multi-module molecules capable of triggering or inhibiting the activation of an immune cell. A CAR will generally comprise a TRACeR sequence as described herein, linker, transmembrane domain and cytoplasmic signaling domain. In some instances, a CAR will include one or more co-stimulatory domains and/or one or more co-inhibitory domains.

A spacer (linker) region links the antigen binding domain to the transmembrane domain. It should be flexible enough to allow the antigen binding domain to orient in different directions to facilitate antigen recognition. The simplest form is the hinge region from an immunoglobulin, e.g. the hinge from any one of IgG1, IgG2a, IgG2b, IgG3, IgG4, particularly the human protein sequences. Alternatives include the CH2CH3 region of immunoglobulin and portions of CD3. For many scFv based constructs, an IgG hinge is effective. In some embodiments the linker comprises the amino acid sequence (G₄S)ₙ where n is 1, 2, 3, 4, 5, etc., and in some embodiments, n is 3.

The CAR transmembrane domain (TM) is frequently derived from type I membrane proteins, such as CD3ζ, CD4, CD8, CD28, etc.

A cytoplasmic signaling domain, such as those derived from the T cell receptor ζ-chain, is employed as part of the CAR in order to produce stimulatory signals for T lymphocyte proliferation and effector function following engagement of the chimeric receptor with the target antigen. Endodomains from co-stimulatory molecules may be included in the cytoplasmic signaling portion of the CAR.

The term “co-stimulatory domain”, refers to a stimulatory domain, typically an endodomain, of a CAR that provides a secondary non-specific activation mechanism through which a primary specific stimulation is propagated. Examples of co-stimulation include antigen nonspecific T cell co-stimulation following antigen specific signaling through the T cell receptor and antigen nonspecific B cell co-stimulation following signaling through the B cell receptor. Co-stimulation, e.g., T cell co-stimulation, and the factors involved have been described in Chen & Flies. Nat Rev Immunol (2013) 13(4):227-42, the disclosure of which are incorporated herein by reference in their entirety. Non-limiting examples of suitable co-stimulatory polypeptides include, but are not limited to, 4-1BB (CD137), CD28, ICOS, OX-40, BTLA, CD27, CD30, GITR, and HVEM.

The term “co-inhibitory domain” refers to an inhibitory domain, typically an endodomain, derived from a receptor that provides secondary inhibition of primary antigen-specific activation mechanisms which prevents co-stimulation. Co-inhibition, e.g., T cell co-inhibition, and the factors involved have been described in Chen & Flies. Nat Rev Immunol (2013) 13(4):227-42 and Thaventhiran et al. J Clin Cell Immunol (2012) S12. In some embodiments, co-inhibitory domains homodimerize. A co-inhibitory domain can be an intracellular portion of a transmembrane protein. Non-limiting examples of suitable co-inhibitory polypeptides include, but are not limited to, CTLA-4 and PD-1.

A first-generation CAR transmits the signal from antigen binding through only a single signaling domain, for example a signaling domain derived from the high-affinity receptor for IgE FcεRlγ, or the CD3ζ chain. The domain contains one or three immunoreceptor tyrosine-based activating motif(s) [ITAM(s)] for antigen-dependent T-cell activation. The ITAM-based activating signal endows T-cells with the ability to lyse the target tumor cells and secret cytokines in response to antigen binding.

Second-generation CARs include a co-stimulatory signal in addition to the CD3ζ signal. Coincidental delivery of the delivered co-stimulatory signal enhances cytokine secretion and antitumor activity induced by CAR-transduced T-cells. The co-stimulatory domain is usually be membrane proximal relative to the CD3ζ domain. Third-generation CARs include a tripartite signaling domain, comprising for example a CD28, CD3ζ, OX40 or 4-1BB signaling region. In fourth generation, or “armored car” CAR T-cells are further gene modified to express or block molecules and/or receptors to enhance immune activity.

CAR variants include split CARs wherein the extracellular portion, the TRACeR sequence and the cytoplasmic signaling domain of a CAR are present on two separate molecules. CAR variants also include ON-switch CARs which are conditionally activatable CARs, e.g., comprising a split CAR wherein conditional hetero-dimerization of the two portions of the split CAR is pharmacologically controlled. CAR molecules and derivatives thereof (i.e., CAR variants) are described, e.g., in PCT Application Nos. US2014/016527, US1996/017060, US2013/063083; Fedorov et al. Sci Transl Med (2013) ;5(215):215ra172; Glienke et al. Front Pharmacol (2015) 6:21; Kakarla & Gottschalk 52 Cancer J (2014) 20(2):151-5; Riddell et al. Cancer J (2014) 20(2):141-4; Pegram et al. Cancer J (2014) 20(2):127-33; Cheadle et al. Immunol Rev (2014) 257(1):91-106; Barrett et al. Annu Rev Med (2014) 65:333-47; Sadelain et al. Cancer Discov (2013) 3(4):388-98; Cartellieri et al., J Biomed Biotechnol (2010) 956304; the disclosures of which are incorporated herein by reference in their entirety.

CAR variants also include bispecific or tandem CARs, which include a secondary CAR binding domain that can either amplify or inhibit the activity of a primary CAR. CAR variants also include inhibitory chimeric antigen receptors (iCARs) which may, e.g., be used as a component of a bispecific CAR system, where binding of a secondary CAR binding domain results in inhibition of primary CAR activation. Tandem CARs (TanCAR) mediate bispecific activation of T cells through the engagement of two chimeric receptors designed to deliver stimulatory or costimulatory signals in response to an independent engagement of two different tumor associated antigens. iCARs use the dual antigen targeting to shut down the activation of an active CAR through the engagement of a second suppressive receptor equipped with inhibitory signaling domains

The dual recognition of different epitopes by two CARs diversely designed to either deliver killing through ζ-chain or costimulatory signals, e.g. through CD28, allows a more selective activation of the reprogrammed T cells by restricting Tandem CARs′ activity to cancer cells expressing simultaneously two antigens rather than one. The potency of delivered signals in engineered T cells will remain below threshold of activation and thus ineffective in absence of the engagement of a costimulatory receptor. The combinatorial antigen recognition enhances selective tumor eradication and protects normal tissues expressing only one antigen from unwanted reactions.

Inhibitory CARs (iCARs) are designed to regulate CAR-T cells’ activity through activation of inhibitory receptors’ signaling modules. This approach combines the activity of two CARs, one of which generates dominant negative signals limiting the responses of CAR-T cells activated by the activating receptor. iCARs can switch off the response of the counteracting activator CAR when bound to a specific antigen expressed only by normal tissues. In this way, iCARs-T cells can distinguish cancer cells from healthy ones, and reversibly block functionalities of transduced T cells in an antigen-selective fashion. CTLA-4 or PD-1 intracellular domains in iCARs trigger inhibitory signals on T lymphocytes, leading to less cytokine production, less efficient target cell lysis, and altered lymphocyte motility.

A TRACeR sequence can be formatted as a “chimeric bispecific binding member”, where the TRACeR sequence provides one of the binding specificities in a chimeric polypeptide having dual specificity to two different binding partners (e.g., two different antigens). Non-limiting examples of chimeric bispecific binding members include bispecific antibodies, bispecific conjugated monoclonal antibodies (mab)₂, bispecific antibody fragments (e.g., F(ab)₂, bispecific scFv, bispecific diabodies, single chain bispecific diabodies, etc.), bispecific T cell engagers (BiTE), bispecific conjugated single domain antibodies, microbodies and mutants thereof, and the like. Non-limiting examples of chimeric bispecific binding members also include those chimeric bispecific agents described in Kontermann. MAbs. (2012) 4(2): 182-197; Stamova et al. Antibodies 2012, 1(2), 172-198; Farhadfar et al. Leuk Res. (2016) 49:13-21; Benjamin et al. Ther Adv Hematol. (2016) 7(3):142-56; Kiefer et al. Immunol Rev. (2016) 270(1):178-92; Fan et al. J Hematol Oncol. (2015) 8:130; May et al. Am J Health Syst Pharm. (2016) 73(1):e6-e13; the disclosures of which are incorporated herein by reference in their entirety.

In some instances, a chimeric bispecific binding member may be a bispecific T cell engager (BiTE). A BiTE is generally made by fusing a specific binding member (e.g., a TRACeR sequence) that binds to a specific MHC/peptide complex, with a second binding domain specific for a T cell molecule such as CD3.

CD3-based bispecific T-cell engager (TCE) is a protein that simultaneously binds through a target antigen on a tumor cell and CD3 on a T-cell to form a TCR-independent artificial immune synapse. Common molecular formats used to create TCE proteins include knob-into-hole format for Fc and light-chain heterodimerization; knob-into-hole format using a common light chain; knob-into-hole triple-chain format; the 2+1 format including a second Fab (Xencor); knob-into-hole triple-chain format; Fab arm exchange; knob-into-hole Cross-MAb 1+1 format; knob into hole CrossMAb; tetravalent scfv Fc fusion; tetravalent HC:LC and scfv fusion; TandAb diabody ; tandem scFv, first generation BiTE®format.

In some instances, a chimeric bispecific binding member may be a CAR T cell adapter. As used herein, by “CAR T cell adapter” is meant an expressed bispecific polypeptide that binds the antigen recognition domain of a CAR and redirects the CAR to a second antigen. Generally, a CAR T cell adapter will have two binding regions, one specific for an epitope on the CAR to which it is directed and a second epitope directed to a binding partner which, when bound, transduces the binding signal activating the CAR. Useful CAR T cell adapters include but are not limited to e.g., those described in Kim et al. J Am Chem Soc. (2015) 137(8):2832-5; Ma et al. Proc Natl Acad Sci U S A. (2016) 113(4):E450-8 and Cao et al. Angew Chem Int Ed Engl. (2016) 55(26):7520-4; the disclosures of which are incorporated herein by reference in their entirety.

Effector CAR-T cells include autologous or allogeneic immune cells having cytolytic activity against a target cell expressing an antigen of interest. The effector cells have cytolytic activity that does not require recognition through the T cell antigen receptor. In some embodiments, a T cell is engineered to express a CAR. The term “T cells” refers to mammalian immune effector cells that may be characterized by expression of CD3 and/or T cell antigen receptor.

In some embodiments, the engineered cells comprise a complex mixture of immune cells, e.g., tumor infiltrating lymphocytes (TILs) isolated from an individual in need of treatment. See, for example, Yang and Rosenberg (2016) Adv Immunol. 130:279-94, “Adoptive T Cell Therapy for Cancer; Feldman et al (2015) Semin Oncol. 42(4):626-39 “Adoptive Cell Therapy-Tumor-Infiltrating Lymphocytes, T-Cell Receptors, and Chimeric Antigen Receptors”; Clinical Trial NCT01174121, “Immunotherapy Using Tumor Infiltrating Lymphocytes for Patients With Metastatic Cancer”; Tran et al. (2014) Science 344(6184)641-645, “Cancer immunotherapy based on mutation-specific CD4+ T cells in a patient with epithelial cancer”.

In other embodiments, the engineered T cell is allogeneic with respect to the individual that is treated, e.g. see clinical trials NCT03121625; NCT03016377; NCT02476734; NCT02746952; NCT02808442. See for review Graham et al. (2018) Cells. 7(10) E155. In some embodiments an allogeneic engineered T cell is fully HLA matched. However not all patients have a fully matched donor and a cellular product suitable for all patients independent of HLA type provides an alternative. A universal ‘off the shelf’ CAR T cell product provides advantages in uniformity of harvest and manufacture.

Allogeneic T cells can be genetically modified to reduce graft v host disease. For example, the TCRαβ receptor can be knocked out by different gene editing techniques. TCRαβ is a heterodimer and both alpha and beta chains need to be present for it to be expressed. A single gene codes for the alpha chain (TRAC), whereas there are 2 genes coding for the beta chain, therefore TRAC loci KO has been deleted for this purpose. A number of different approaches have been used to accomplish this deletion, e.g. CRISPR/Cas9; meganuclease; engineered I-Crel homing endonuclease, etc. See, for example, Eyquem et al. (2017) Nature 543:113-117, in which the TRAC coding sequence is replaced by the CAR coding sequence; and Georgiadis et al. (2018) Mol. Ther. 26:1215-1227, which linked CAR expression with TRAC disruption by clustered regularly interspaced short palindromic repeats (CRISPR)/Cas9 without directly incorporating the CAR into the TRAC loci. An alternative strategy to prevent GVHD modifies CAR-T cells to express an inhibitor of TCRαβ signaling, for example using a truncated form of CD3ζ as a TCR inhibitory molecule.

Allogeneic T cells may be administered in combination with intensification of lymphodepletion to allow CAR-T cells to expand and clear malignant cells prior to host immune recovery, e.g. by administration of Alemtuzumab (monoclonal anti-CD52), purine analogs, etc. The allogeneic T cells may be modified for resistance to Alemtuzumab, and currently in clinical trials. Gene editing has also been used to prevent expression of HLA class I molecules on CAR-T cells, e.g. by deletion of β2-microglobulin, see NCT03166878.

In addition to modifying T cells, induced pluripotent stem (iPS) CAR-T cells can provide a source of allogeneic CAR-T cells. For example, transducing donor T cells with reprogramming factors can restore pluripotency, and are then re-differentiated to T effector cells.

T cells for engineering as described above collected from a subject or a donor may be separated from a mixture of cells by techniques that enrich for desired cells, or may be engineered and cultured without separation. An appropriate solution may be used for dispersion or suspension. Such solution will generally be a balanced salt solution, e.g. normal saline, PBS, Hank’s balanced salt solution, etc., conveniently supplemented with fetal calf serum or other naturally occurring factors, in conjunction with an acceptable buffer at low concentration, generally from 5-25 mM. Convenient buffers include HEPES, phosphate buffers, lactate buffers, etc.

Expression construct: A polynucleotide encoding a TRACeR sequence or a polypeptide comprising a TRACeR sequence, e.g. CAR, antibody, Fc fusion, drug conjugate, etc. can be provided on an expression vector. The nucleic acid encoding a TRACeR sequence is inserted into a vector for expression and/or integration. Many such vectors are available. The vector components generally include, but are not limited to, one or more of the following: an origin of replication, one or more marker genes, an enhancer element, a promoter, and a transcription termination sequence. Vectors include viral vectors, plasmid vectors, integrating vectors, and the like.

For example, a CAR coding sequence may be introduced into the site of the endogenous T cell receptor, e.g. TRAC gene, e.g., using CRISPR technology (see, for example Eyquem et al. (2017) Nature 543:113-117; Ren et al. (2017) Protein & Cell 1-10; Ren et al. (2017) Oncotarget 8(10):17002-17011). CRISPR/Cas9 system can be directly applied to human cells by transfection with a plasmid that encodes Cas9 and sgRNA. The viral delivery of CRISPR components has been extensively demonstrated using lentiviral and retroviral vectors. Gene editing with CRISPR encoded by non-integrating virus, such as adenovirus and adenovirus-associated virus (AAV), has also been reported. Recent discoveries of smaller Cas proteins have enabled and enhanced the combination of this technology with vectors that have gained increasing success for their safety profile and efficiency, such as AAV vectors.

Expression vectors may contain a selection gene, also termed a selectable marker. This gene encodes a protein necessary for the survival or growth of transformed host cells grown in a selective culture medium. Host cells not transformed with the vector containing the selection gene will not survive in the culture medium. Typical selection genes encode proteins that (a) confer resistance to antibiotics or other toxins, e.g., ampicillin, neomycin, methotrexate, or tetracycline, (b) complement auxotrophic deficiencies, or (c) supply critical nutrients not available from complex media.

Nucleic acids are “operably linked” when placed into a functional relationship with another nucleic acid sequence. For example, DNA for a signal sequence is operably linked to DNA for a polypeptide if it is expressed as a preprotein that signals the secretion of the polypeptide; a promoter or enhancer is operably linked to a coding sequence if it affects the transcription of the sequence; and a ribosome binding site is operably linked to a coding sequence if it is positioned so as to facilitate translation. Generally, “operably linked” means that the DNA sequences being linked are contiguous, and, in the case of a secretory leader, contiguous and in reading phase. However, enhancers do not have to be contiguous.

Expression vectors will contain a promoter that is recognized by the host organism and is operably linked to the TRACeR coding sequence. Promoters are untranslated sequences located upstream (5′) to the start codon of a structural gene (generally within about 100 to 1000 bp) that control the transcription and translation of particular nucleic acid sequences to which they are operably linked. Such promoters typically fall into two classes, inducible and constitutive. Inducible promoters are promoters that initiate increased levels of transcription from DNA under their control in response to a change in culture conditions, e.g., the presence or absence of a nutrient or a change in temperature. A large number of promoters recognized by a variety of potential host cells are well known.

Transcription from vectors in mammalian host cells may be controlled, for example, by promoters obtained from the genomes of viruses such as polyoma virus, fowlpox virus, adenovirus (such as Adenovirus 2), bovine papilloma virus, avian sarcoma virus, cytomegalovirus, a retrovirus LTR (such as murine stem cell virus), hepatitis-B virus and Simian Virus 40 (SV40), from heterologous mammalian promoters, e.g., the actin promoter, PGK (phosphoglycerate kinase), or an immunoglobulin promoter, or from heat-shock promoters, provided such promoters are compatible with the host cell systems. The early and late promoters of the SV40 virus are conveniently obtained as an SV40 restriction fragment that also contains the SV40 viral origin of replication.

Transcription by higher eukaryotes may be increased by inserting an enhancer sequence into the vector. Enhancers are cis-acting elements of DNA, usually about from 10 to 300 bp in length, which act on a promoter to increase its transcription. Enhancers are relatively orientation and position independent, having been found 5′ and 3′ to the transcription unit, within an intron, as well as within the coding sequence itself. Many enhancer sequences are now known from mammalian genes (globin, elastase, albumin, α-fetoprotein, and insulin). Typically, however, one will use an enhancer from a eukaryotic virus. Examples include the SV40 enhancer on the late side of the replication origin, the cytomegalovirus early promoter enhancer, the polyoma enhancer on the late side of the replication origin, and adenovirus enhancers. The enhancer may be spliced into the expression vector at a position 5′ or 3′ to the coding sequence, but is preferably located at a site 5′ from the promoter.

Expression vectors for use in eukaryotic host cells will also contain sequences necessary for the termination of transcription and for stabilizing the mRNA. Such sequences are commonly available from the 5′ and, occasionally 3′, untranslated regions of eukaryotic or viral DNAs or cDNAs. Construction of suitable vectors containing one or more of the above-listed components employs standard techniques.

Suitable host cells for cloning or expressing a TRACeR sequence are the prokaryotic and yeast, or other eukaryotic cells described above. Examples of useful mammalian host cell lines are mouse L cells (L-M[TK-], ATCC#CRL-2648), monkey kidney CV1 line transformed by SV40 (COS-7, ATCC CRL 1651); human embryonic kidney line (293 or 293 cells subcloned for growth in suspension culture; baby hamster kidney cells (BHK, ATCC CCL 10); Chinese hamster ovary cells/-DHFR (CHO); mouse Sertoli cells (TM4); monkey kidney cells (CV1 ATCC CCL 70); African green monkey kidney cells (VERO-76, ATCC CRL-1 587); human cervical carcinoma cells (HELA, ATCC CCL 2); canine kidney cells (MDCK, ATCC CCL 34); buffalo rat liver cells (BRL 3A, ATCC CRL 1442); human lung cells (W138, ATCC CCL 75); human liver cells (Hep G2, HB 8065); mouse mammary tumor (MMT 060562, ATCC CCL51); TRI cells; MRC 5 cells; FS4 cells; and a human hepatoma line (Hep G2).

Host cells, including T cells, stem cells, etc. can be transfected with the above-described expression vectors for TRACeR construct expression. Cells may be cultured in conventional nutrient media modified as appropriate for inducing promoters, selecting transformants, or amplifying the genes encoding the desired sequences. Mammalian host cells may be cultured in a variety of media. Commercially available media such as Ham’s F10 (Sigma), Minimal Essential Medium ((MEM), Sigma), RPMI 1640 (Sigma), and Dulbecco’s Modified Eagle’s Medium ((DMEM), Sigma) are suitable for culturing the host cells. Any of these media may be supplemented as necessary with hormones and/or other growth factors (such as insulin, transferrin, or epidermal growth factor), salts (such as sodium chloride, calcium, magnesium, and phosphate), buffers (such as HEPES), nucleosides (such as adenosine and thymidine), antibiotics, trace elements, and glucose or an equivalent energy source. Any other necessary supplements may also be included at appropriate concentrations that would be known to those skilled in the art. The culture conditions, such as temperature, pH and the like, are those previously used with the host cell selected for expression, and will be apparent to the ordinarily skilled artisan.

The terms “polypeptide,” “peptide” and “protein” are used interchangeably herein to refer to a polymer of amino acid residues. The terms also apply to amino acid polymers in which one or more amino acid residue is an artificial chemical mimetic of a corresponding naturally occurring amino acid, as well as to naturally occurring amino acid polymers and non-naturally occurring amino acid polymer.

The term “sequence identity,” as used herein in reference to polypeptide or DNA sequences, refers to the subunit sequence identity between two molecules. When a subunit position in both of the molecules is occupied by the same monomeric subunit (e.g., the same amino acid residue or nucleotide), then the molecules are identical at that position. The similarity between two amino acid or two nucleotide sequences is a direct function of the number of identical positions. In general, the sequences are aligned so that the highest order match is obtained. If necessary, identity can be calculated using published techniques and widely available computer programs, such as the GCS program package (Devereux et al., Nucleic Acids Res. 12:387, 1984), BLASTP, BLASTN, FASTA (Atschul et al., J. Molecular Biol. 215:403, 1990).

By “protein variant” or “variant protein” or “variant polypeptide” herein is meant a protein that differs from a wild-type protein by virtue of at least one amino acid modification. The parent polypeptide may be a naturally occurring or wild-type (WT) polypeptide, or may be a modified version of a WT polypeptide. Variant polypeptide may refer to the polypeptide itself, a composition comprising the polypeptide, or the amino sequence that encodes it. Preferably, the variant polypeptide has at least one amino acid modification compared to the parent polypeptide, e.g. from about one to about ten amino acid modifications, and preferably from about one to about five amino acid modifications compared to the parent.

By “parent polypeptide”, “parent protein”, “precursor polypeptide”, or “precursor protein” as used herein is meant an unmodified polypeptide that is subsequently modified to generate a variant. A parent polypeptide may be a wild-type (or native) polypeptide, or a variant or engineered version of a wild-type polypeptide. Parent polypeptide may refer to the polypeptide itself, compositions that comprise the parent polypeptide, or the amino acid sequence that encodes it.

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, gamma-carboxyglutamate, and O-phosphoserine. “Amino acid analogs” refers to compounds that have the same basic chemical structure as a naturally occurring amino acid, i.e., an α-carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. “Amino acid mimetics” refers to chemical compounds that have a structure that is different from the general chemical structure of an amino acid, but that function in a manner similar to a naturally occurring amino acid.

Amino acid modifications disclosed herein may include amino acid substitutions, deletions and insertions, particularly amino acid substitutions. Variant proteins may also include conservative modifications and substitutions at other positions of the cytokine and/or receptor (e.g., positions other than those involved in the affinity engineering). Such conservative substitutions include those described by Dayhoff in The Atlas of Protein Sequence and Structure 5 (1978), and by Argos in EMBO J., 8:779-785 (1989). For example, amino acids belonging to one of the following groups represent conservative changes: Group I: Ala, Pro, Gly, Gln, Asn, Ser, Thr; Group II: Cys, Ser, Tyr, Thr; Group III: Val, Ile, Leu, Met, Ala, Phe; Group IV: Lys, Arg, His; Group V: Phe, Tyr, Trp, His; and Group VI: Asp, Glu. Further, amino acid substitutions with a designated amino acid may be replaced with a conservative change.

The term “isolated” refers to a molecule that is substantially free of its natural environment. For instance, an isolated protein is substantially free of cellular material or other proteins from the cell or tissue source from which it is derived. The term refers to preparations where the isolated protein is sufficiently pure to be administered as a therapeutic composition, or at least 70% to 80% (w/w) pure, more preferably, at least 80%-90% (w/w) pure, even more preferably, 90-95% pure; and, most preferably, at least 95%, 96%, 97%, 98%, 99%, or 100% (w/w) pure. A “separated” compound refers to a compound that is removed from at least 90% of at least one component of a sample from which the compound was obtained. Any compound described herein can be provided as an isolated or separated compound.

The terms “subject,” “individual,” and “patient” are used interchangeably herein to refer to a mammal being assessed for treatment and/or being treated. In some embodiments, the mammal is a human. The terms “subject,” “individual,” and “patient” encompass, without limitation, individuals having a disease. Subjects may be human, but also include other mammals, particularly those mammals useful as laboratory models for human disease, e.g., mice, rats, etc.

The effect of treatment can be prophylactic in terms of completely or partially preventing infection. Those in need of treatment include those already infected (e.g., those with infection, those with an infection, etc.) as well as those in which prevention is desired (e.g., those with increased susceptibility to infection, those with an increased likelihood of infection, those suspected of having infection, those suspected of harboring an infection, etc.).

A therapeutic treatment is one in which the subject is infected prior to administration and a prophylactic treatment is one in which the subject is not infected prior to administration. In some embodiments, the subject has an increased likelihood of becoming infected or is suspected of being infected prior to treatment. In some embodiments, the subject is suspected of having an increased likelihood of becoming infected.

As used herein, the term “infection” refers to any state in at least one cell of an organism (i.e., a subject) is infected by a pathogen. A pathogen may be an intracellular pathogen, e.g. certain bacteria, protozoans, and viruses.

Viruses include those that infect, e.g. farm animals including horses, cattle, sheep, pigs, chickens, turkeys, etc., domestic animals including dogs and cats; and viruses that infect humans. In some embodiments a is an RNA virus. An RNA virus is a virus that has RNA (ribonucleic acid) as its genetic material. This nucleic acid is usually single-stranded RNA (ssRNA) but may be double-stranded RNA (dsRNA). Human diseases caused by RNA viruses include AIDS, Ebola hemorrhoragic fever, SARS, influenza, hepatitis C, West Nile fever, polio, and measles.

The ICTV classifies RNA viruses as those that belong to Group III, Group IV or Group V of the Baltimore classification system of classifying viruses and does not consider viruses with DNA intermediates in their life cycle as RNA viruses. Viruses with RNA as their genetic material but that include DNA intermediates in their replication cycle are retroviruses, and comprise Group VI of the Baltimore classification. Notable human retroviruses include HIV-1 and HIV-2, the cause of the disease AIDS. For the purposes of the present invention, an RNA virus is one that is within Group III, IV, V or VI unless otherwise indicated.

The double-stranded (ds)RNA viruses represent a diverse group of viruses that vary widely in host range, genome segment number, and virion organization. Members of this group include the rotaviruses and picobirnaviruses. The clades include the Caliciviridae, Flaviviridae, and Picornaviridae families, and a second that includes the Alphatetraviridae, Birnaviridae and Cystoviridae, Nodaviridae, and Permutotretraviridae families. Double-stranded RNA viruses (Group III) contain from one to a dozen different RNA molecules, each coding for one or more viral proteins.

RNA viruses can be further classified according to the sense or polarity of their RNA into negative-sense and positive-sense, or ambisense RNA viruses. Positive-sense ssRNA viruses (Group IV) have their genome directly utilized as if it were mRNA, with host ribosomes translating it into a single protein that is modified by host and viral proteins to form the various proteins needed for replication. One of these includes RNA-dependent RNA polymerase (RNA replicase), which copies the viral RNA to form a double-stranded replicative form. In turn this directs the formation of new virions. Viruses in this group include I. Bymoviruses, comoviruses, nepoviruses, nodaviruses, picornaviruses, potyviruses, sobemoviruses and a subset of luteoviruses (beet western yellows virus and potato leafroll virus)-the picorna like group (Picornavirata); II. Carmoviruses, dianthoviruses, flaviviruses, pestiviruses, tombusviruses, hepatitis C virus and a subset of luteoviruses (barley yellow dwarf virus)-the flavi like group (Flavivirata); III. Alphaviruses, carlaviruses, furoviruses, hordeiviruses, potexviruses, rubiviruses, tobraviruses, tricornaviruses, tymoviruses and hepatitis E virus-the alpha like group (Rubivirata). Alphaviruses and flaviviruses can be separated into two families-the Togaviridae and Flaviridae. Coronavirus are of particular interest, e.g. SARS-CoV1, SARS-CoV2; MERS-CoV, etc.

Negative-sense ssRNA viruses (Group V) must have their genome copied by an RNA replicase to form positive-sense RNA. The positive-sense RNA molecule then acts as viral mRNA, which is translated into proteins by the host ribosomes. The resultant protein goes on to direct the synthesis of new virions, such as capsid proteins and RNA replicase, which is used to produce new negative-sense RNA molecules. Group V-negative-sense ssRNA viruses include one order and eight families in this group. The group includes a number of clinically relevant pathogens. Bornaviridae-Borna disease virus; Family Filoviridae-includes Ebola virus, Marburg virus; Family Paramyxoviridae-includes Measles virus, Mumps virus, Nipah virus, Hendra virus, RSV and NDV; Family Rhabdoviridae-includes Rabies virus; Family Nyamiviridae-includes Nyavirus; Family Arenaviridae-includes Lassa virus; Family Bunyaviridae-includes Hantavirus, Crimean-Congo hemorrhagic fever; Family Ophioviridae; Family Orthomyxoviridae-includes Influenza viruses; Genus Deltavirus-includes Hepatitis D virus; Genus Dichorhavirus; Genus Emaravirus; Genus Nyavirus-includes Nyamanini and Midway viruses; Genus Tenuivirus; Genus Varicosavirus

Retroviruses (Group VI) have a single-stranded RNA genome although they use DNA intermediates to replicate. Reverse transcriptase, a viral enzyme that comes from the virus itself after it is uncoated, converts the viral RNA into a complementary strand of DNA, which is copied to produce a double-stranded molecule of viral DNA. After this DNA is integrated into the host genome using the viral enzyme integrase, expression of the encoded genes may lead to the formation of new virions. Included in retroviruses are the lentiviruses, e.g. HIV-1 and HIV-2.

The term “sample” with reference to a patient encompasses blood and other liquid samples of biological origin, solid tissue samples such as a biopsy specimen or tissue cultures or cells derived therefrom and the progeny thereof. The term also encompasses samples that have been manipulated in any way after their procurement, such as by treatment with reagents; washed; or enrichment for certain cell populations, such as diseased cells. The definition also includes samples that have been enriched for particular types of molecules, e.g., nucleic acids, polypeptides, etc. The term “biological sample” encompasses a clinical sample, and also includes tissue obtained by surgical resection, tissue obtained by biopsy, cells in culture, cell supernatants, cell lysates, tissue samples, organs, bone marrow, blood, plasma, serum, and the like. A “biological sample” includes a sample obtained from a patient’s diseased cell, e.g., a sample comprising polynucleotides and/or polypeptides that is obtained from a patient’s diseased cell (e.g., a cell lysate or other cell extract comprising polynucleotides and/or polypeptides); and a sample comprising diseased cells from a patient. A biological sample comprising a diseased cell from a patient can also include non-diseased cells.

The term “diagnosis” is used herein to refer to the identification of a molecular or pathological state, disease or condition in a subject, individual, or patient.

The term “prognosis” is used herein to refer to the prediction of the likelihood of death or disease progression, including recurrence, spread, and drug resistance, in a subject, individual, or patient. The term “prediction” is used herein to refer to the act of foretelling or estimating, based on observation, experience, or scientific reasoning, the likelihood of a subject, individual, or patient experiencing a particular event or clinical outcome. In one example, a physician may attempt to predict the likelihood that a patient will survive.

As used herein, the terms “treatment,” “treating,” and the like, refer to administering an agent, or carrying out a procedure, for the purposes of obtaining an effect on or in a subject, individual, or patient. The effect may be prophylactic in terms of completely or partially preventing a disease or symptom thereof and/or may be therapeutic in terms of effecting a partial or complete cure for a disease and/or symptoms of a disease. “Treatment,” as used herein, may include treatment of cancer in a mammal, particularly in a human, and includes: (a) inhibiting the disease, i.e., arresting its development; and (b) relieving the disease or its symptoms, i.e., causing regression of the disease or its symptoms.

Treating may refer to any indicia of success in the treatment or amelioration or prevention of a disease, including any objective or subjective parameter such as abatement; remission; diminishing of symptoms or making the disease condition more tolerable to the patient; slowing in the rate of degeneration or decline; or making the final point of degeneration less debilitating. The treatment or amelioration of symptoms can be based on objective or subjective parameters; including the results of an examination by a physician. Accordingly, the term “treating” includes the administration of engineered cells to prevent or delay, to alleviate, or to arrest or inhibit development of the symptoms or conditions associated with disease or other diseases. The term “therapeutic effect” refers to the reduction, elimination, or prevention of the disease, symptoms of the disease, or side effects of the disease in the subject.

As used herein, a “therapeutically effective amount” refers to that amount of the therapeutic agent, e.g. an infusion of engineered T cells, and antibody construct, etc., sufficient to treat or manage a disease or disorder. A therapeutically effective amount may refer to the amount of therapeutic agent sufficient to delay or minimize the onset of disease, e.g., to delay or minimize the growth and spread of cancer. A therapeutically effective amount may also refer to the amount of the therapeutic agent that provides a therapeutic benefit in the treatment or management of a disease. Further, a therapeutically effective amount with respect to a therapeutic agent of the invention means the amount of therapeutic agent alone, or in combination with other therapies, that provides a therapeutic benefit in the treatment or management of a disease.

As used herein, the term “dosing regimen” refers to a set of unit doses (typically more than one) that are administered individually to a subject, typically separated by periods of time. In some embodiments, a given therapeutic agent has a recommended dosing regimen, which may involve one or more doses. In some embodiments, a dosing regimen comprises a plurality of doses each of which are separated from one another by a time period of the same length; in some embodiments, a dosing regimen comprises a plurality of doses and at least two different time periods separating individual doses. In some embodiments, all doses within a dosing regimen are of the same unit dose amount. In some embodiments, different doses within a dosing regimen are of different amounts. In some embodiments, a dosing regimen comprises a first dose in a first dose amount, followed by one or more additional doses in a second dose amount different from the first dose amount. In some embodiments, a dosing regimen comprises a first dose in a first dose amount, followed by one or more additional doses in a second dose amount same as the first dose amount. In some embodiments, a dosing regimen is correlated with a desired or beneficial outcome when administered across a relevant population (i.e., is a therapeutic dosing regimen).

“In combination with”, “combination therapy” and “combination products” refer, in certain embodiments, to the concurrent administration to a patient of the engineered proteins and cells described herein in combination with additional therapies, e.g. surgery, radiation, chemotherapy, and the like. When administered in combination, each component can be administered at the same time or sequentially in any order at different points in time. Thus, each component can be administered separately but sufficiently closely in time so as to provide the desired therapeutic effect.

“Concomitant administration” means administration of one or more components, such as engineered proteins and cells, known therapeutic agents, etc. at such time that the combination will have a therapeutic effect. Such concomitant administration may involve concurrent (i.e. at the same time), prior, or subsequent administration of components. A person of ordinary skill in the art would have no difficulty determining the appropriate timing, sequence and dosages of administration.

The use of the term “in combination” does not restrict the order in which prophylactic and/or therapeutic agents are administered to a subject with a disorder. A first prophylactic or therapeutic agent can be administered prior to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks 6 weeks, 8 weeks, or 12 weeks before), concomitantly with, or subsequent to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks after) the administration of a second prophylactic or therapeutic agent to a subject with a disorder.

### Polypeptide and Polynucleotide Compositions

A library composition of the disclosure comprises designed TRACeR binding proteins, where the polypeptides in the library comprise an engineered peptide recognition sequence (PRE) and MHC interacting surfaces. The scaffold of the TRACeR binding protein localizes antigen recognition to a single structural element, and provides specificity and affinity with an engineered peptide recognition element (PRE) loop.

In the design of TRACeR sequence formulas, variations of design choices are contemplated as set forth below. In the design of a TRACeR sequence formula, the MAM protein (SEQ ID NO:1) is a starting point. The protein is cleaved between residue 124 and 125, where residues 125-213 are not further used. In some embodiments the cleavage site can be immediately after residue 120, 121, 123, 125, 126, 127, where the correspondingly truncated or extended terminal sequence is used in the TRACeR sequence formula.

(SEQ ID NO:1)MKLRVENPKKAQKHFVQNLNNVVFTNKELEDIYDLSN
KEETKEVLKLFKLKVNQFYRHAFGIVNDYNGLLEYKEIFNMMFLKLSVVF
DTQRKEANNVEQIKRNIAILDEIMAKADNDLSYFISQNKNFQELWDKAVK
LTKEMKIKLKGQKLDLRDGEVAINKVRELFGSDKNVKESWWFRSLLVKGV
YLIKRYYEGDIELKTTSDFAKAVFED

TRACeR proteins generally comprise 3 regions of interests for engineering: (A) the PRE, (B) scaffold interfacial positions, and (C) a short helical connector. The positioning of these regions disclosed below is made with by numbering with reference relative to the MAM crystal structure sequence (Uniprot B3PLU9).

The PRE loop (A) refers to residues 1 and 20, where any residue can be randomized in this region. The region can be shortened by deletion of any residue, provided that the length remain at least about 8, at least 9, at least about 10, at least 11, at least about 12 amino acids. In some embodiments residues between 10-19 are sites for randomization, e.g. as shown in SEQ ID NO:3, 4 and 6.

Stability of the PRE loop requires the presence of a stabilizing disulfide bond, which is provided by the substitution of two residues with cysteines. One of the cysteines is positioned at any site in the PRE loop. A second cysteine is outside of the PRE loop, in helix3 (residues 71-93) or helix4 (residues 96-124), and may be, for example, at residue 73, 74, 75, 77, 78, 115, 116, 117, 119, 120. In certain embodiments, the cysteines are positioned at residues 5 and 119.

Scaffold interfacial positions, (B), are outside of the PRE, and include residues 22, 77, 78, 80, 81, 82, 85, 87, 88, 89, 92, 95, 97, 98, 99, 101, 102, 105, 106, 108, 109, 113, 116, 117, 120. These residues can be randomized, or substituted with a limited set of amino acids, to alter the MHC binding specificity of the protein.

The helical connector (C) provides amino acid substitutions in the “alpha-alpha loop”, with numbering relative to SEQ ID NO:1, residues 67-72, where residues 67-72 NGLLEY are substituted with the two amino acids GD. The redesign of (C) is optional.

Regions (A) and (C) are considered together, and are relevant for determining binding specificity and affinity to an MHC/peptide complex of a TRACeR protein, and are randomized or modified as discussed herein. (A) can be evolved separately from (B) or (C), but in the context of a specific background B/C sequence. (B) is where MHC-I and MHC-II binders differ, and can be modified with the amino acid substitutions described below with reference to SEQ ID NO:5.

The PRE sequence is randomized in according to the sequence formulas provided herein and may comprise, for example, 1, 2, 3, 4, 5, 6, 7, 8, 9, 10 or more randomized residues. In some embodiments the number of randomized residues is from 1 to 10, 1 to 8, 1 to 7, 1 to 6, 1 to 5, 1 to 4, 1 to 3, 1 to 2, 2 to 6, 2 to 5, 2 to 4, 2 to 3; 3 to 5, 3 to 4; etc. Any randomized residue may be located within a contiguous region of randomized residues, or may be located at non-contiguous residues within the larger “loop” sequence, as defined herein. A randomized residue can be any amino acid, or can be a limited set of amino acids.

Sequences provided herein that include randomized residues as discussed above may be referred to as “sequence formulas”, which include, without limitation, any of SEQ ID NO:2, 3, 4, 5, 6. The TRACeR sequence formulas provided herein define a plurality of polypeptides, where the number of different polypeptides within a formula is defined by the number of randomized residues, and whether those residues are any amino acid, or a limited set of amino acids.

An aspect of the engineered proteins is the introduction of a stabilizing disulfide bond, which is present in both sequence formulas for binding to Class II MHC/peptide complexes and for binding to MHC Class I MHC/peptide complexes. The disulfide bond is formed by substitution of cysteine for two amino acids in the sequence. An N-terminal cysteine can be part of the peptide recognition loop, and a second cysteine is on the scaffold. While exemplary cysteine substitutions are provided in the sequence formulas disclosed herein, these are not limiting. For example, and without limitation, positions for cysteine substitution, using numbering of positions relative to SEQ ID NO:1 residues 1-124, cysteine substitutions can be made at residues 4 and 115; 5 and 119; 16 and 77; 17 and 74.

In some embodiments a TRACeR sequence formula for MHC Class II/peptide selection comprises a polypeptide having at least 85% sequence identity to SEQ ID NO:1, residues 1-124, and further comprising substitution of two amino acids for cysteine to provide a disulfide bond; a randomized region of from 1 to 10 amino acids; and a substitution of amino acids GD for positions 67-72 wherein a polypeptide in the population binds to a Class II MHC protein with an affinity of less than 10⁶M.

In some embodiments a TRACeR library is provided for screening MHC Class II/peptide complexes. In some embodiments and without limitation as to the variations disclosed herein, a TRACeR library has a sequence according to the formula set forth in any of SEQ ID NO:2, SEQ ID NO:3 or SEQ ID NO:4. Degeneracy is generated at the variable residues indicated in the sequence formulas to create a library of protein sequences, which library can be screened for specific peptides presented in the Class II HLA context. In some embodiments, for example and without limitation, SEQ ID NO:3, residues 5 and 119; SEQ ID NO:4, residues 5 and 115, the TRACeR polypeptides comprise cysteine residues that form a stabilizing disulfide bond. Individual TRACeR proteins with specificity for an MHC/peptide complex of interest are identified by screening, and can be further used in a format that provides for therapeutic efficacy. SEQ ID NO:4 is of particular interest for this purpose. In some such embodiments the HLA class II protein is human HLA-DR1.

SEQ ID NO:3MKLRCENPKKAXXXXXQNLNNVVFTNKELEDIYDLSNKE
ETKEVLKLFKLKVNQFYRHAFGIVNDYNGLLEYKEIFNMMFLKLSVVFDT
QRKEANNVEQIKRNIAILDEIMAKADNDLCYFISQ

In some embodiments, X is any amino acid.

In some embodiments, residues 12-16 of SEQ ID NO:3 (the randomized residues) are any of SEQ ID NO:17-SEQ ID NO:40, as shown below:

For HA specific binding:

| | | | | | |
|---|---|---|---|---|---|
| E | V | H | M | V | SEQ ID NO:17 |
| D | N | H | L | V | SEQ ID NO:18 |
| E | T | H | W | V | SEQ ID NO:19 |
| E | S | H | L | V | SEQ ID NO:20 |
| E | R | H | L | L | SEQ ID NO:21 |
| D | N | H | L | V | SEQ ID NO:22 |
| E | V | H | W | V | SEQ ID NO:23 |
| D | W | H | L | V | SEQ ID NO:24 |

For NYESO-1 specific binding:

| | | | | | |
|---|---|---|---|---|---|
| F | R | W | G | G | SEQ ID NO:25 |
| V | R | W | G | G | SEQ ID NO:26 |
| V | R | W | G | G | SEQ ID NO:27 |
| V | K | W | G | G | SEQ ID NO:28 |
| Y | V | L | G | V | SEQ ID NO:29 |
| G | R | F | G | V | SEQ ID NO:30 |
| Y | R | W | G | G | SEQ ID NO:31 |
| V | R | W | G | G | SEQ ID NO:32 |

For CLIP specific binding:

| | | | | | |
|---|---|---|---|---|---|
| S | T | Y | L | A | SEQ ID NO:33 |
| S | I | F | L | A | SEQ ID NO:34 |
| Q | L | F | L | A | SEQ ID NO:35 |
| V | V | F | L | V | SEQ ID NO:36 |
| V | L | F | L | V | SEQ ID NO:37 |
| A | V | Y | L | A | SEQ ID NO:38 |
| A | V | Y | G | A | SEQ ID NO:39 |
| T | A | Y | G | A | SEQ ID NO:40 |

SEQ ID NO:4MKLRCENPKKAXXXXXXXXNNVVFTNKELEDIYDLSNKE
ETKEVLKLFKLKVNQFYRHAFGIVNDYGDKEIFNMMFLKLSVVFDTQRKE
ANNVEQIKRNIAILDEIMAKADNDLCYFISQ

In some embodiments a TRACeR library is provided for screening MHC Class I/peptide complexes. To develop a sequence formula for binding to a Class I MHC allele, it is first necessary to screen a library for specific binding to a Class I allele of interest. In some embodiments this sequence formula is as set forth in SEQ ID NO:5:

| 1 | 2 | 3 | 4 | 5 | 6 | 7 | 8 | 9 | 10 | 11 | 12 | 13 | 14 | 15 | 16 | 17 | 18 | 19 | 20 |
|---|---|---|---|---|---|---|---|---|---|---|---|---|---|---|---|---|---|---|---|
| M | K | L | R | C | E | N | P | K | K | A | X | X | X | N | X | Q | N | L | N |
| 21 | 22 | 23 | 24 | 25 | 26 | 27 | 28 | 29 | 30 | 31 | 32 | 33 | 34 | 35 | 36 | 37 | 38 | 39 | 40 |
| N | V | V | F | T | N | K | E | L | E | D | I | Y | D | L | S | N | K | E | E |
| 41 | 42 | 43 | 44 | 45 | 46 | 47 | 48 | 49 | 50 | 51 | 52 | 53 | 54 | 55 | 56 | 57 | 58 | 59 | 60 |
| T | K | E | V | L | K | L | F | K | L | K | V | N | Q | F | Y | R | H | A | F |
| 61 | 62 | 63 | 64 | 65 | 66 | 67 | 68 | 69 | 70 | 71 | 72 | 73 | 74 | 75 | 76 | 77 | 78 | 79 | 80 |
| G | I | V | N | D | Y | G | D | K | E | I | F | N | M | M | F | X | X | L | W |
| 81 | 82 | 83 | 84 | 85 | 86 | 87 | 88 | 89 | 90 | 91 | 92 | 93 | 94 | 95 | 96 | 97 | 98 | 99 | 100 |
| X | V | F | X | S | Q | X | X | X | A | N | N | V | E | X | I | K | X | N | I |
| 101 | 102 | 103 | 104 | 105 | 106 | 107 | 108 | 109 | 110 | 111 | 112 | 113 | 114 | 115 | 116 | 117 | 118 | 119 | 120 |
| X | X | L | D | W | I | M | A | E | A | D | N | D | L | C | Y | F | I | S | Q |

In some embodiments, any of the X positions, i.e. at residues 12, 13, 14, 77, 78, 81, 84, 87, 88, 89, 95, 98, 101, 102 are any amino acid, or as defined below. These positions correspond to interfacial residues.

Positions 12, 13, 14 are within the PRE sequence, and may be any amino acid.

In some embodiments, a polypeptide comprises one or more of randomized residues as follows (numbering in reference to SEQ ID NO:5), and may comprise all the randomized residues as follows:
residue 9 is selected from K/D/V
residue 10 is selected from K/E/H
residue 12 is any amino acid other than C
residue 13 is any amino acid other than C
residue 14 is any amino acid other than C
residue 15 is selected from N/D/F/R
residue 16 is selected from A/L/M
residue 19 is selected from L/E/F/N/Q/W/Y
residue 22 is selected from V/L
residue 77 is selected from E/L/M/A/I/N/W
residue 78 is selected from L/M/F
residue 80 is selected from W/F/I/K/L/Y
residue 81 is selected from A/E/I/L/M/Q/S/T/V/W/Y
residue 82 is selected from D/I/L/M/Q/R/S/T/V/W
residue 85 is selected from S/I/L/M/T
residue 87 is selected from R/W/H/K/M/T
residue 88 is selected from F/Y/I/L/R/V/W
residue 89 is selected from N/S/A/F/W/Y
residue 92 is selected from D/N
residue 95 is selected from L/M/Q/V
residue 97 is selected from K/E
residue 98 is selected from F/A/L/T/W
residue 101 is selected from K/R/E/F
residue 102 is selected from M/F/I/L/T/V
residue 105 is selected from W/F/L/M
residue 108 is selected from A/K/R
residue 109 is selected from E/F/K/M/Q/V

In some embodiments a TRACeR library for MHC Class I screening has a sequence formula according to SEQ ID NO:6, which finds use in, for example, screening for MHC Class I/peptide complexes where the Class I allele is an HLA A*02 allele some such embodiments the MHC protein is human HLA-A*02.

(SEQ ID NO:6)MKLRCENPKXXXXXXXQNLNNVVFTNKELEDIYDLSN
KEETKEVLKLFKLKVNQFYRHAFGIVNDYGDKEIFNMMFMLLWRVFRSQR
IDANNVELIKFNIRVLDWIMAEADNDLCYFISQ.

X may be any amino acid.

SEQ ID NO:16 is exemplary for a specific binding protein isolated from a library having the sequence formula of SEQ ID NO:6.

In an embodiment, a TRACeR sequence is covalently linked, e.g. as a single polypeptide fused in frame to an effector polypeptide of a CAR, to an Fc sequence, etc. In an embodiment a TRACeR sequence is provided as a polypeptide linked to an immunoglobulin effector sequence, for example an Fc sequence.

Also provided are isolated nucleic acids encoding TRACeR sequences and constructs thereof, vectors and host cells comprising the nucleic acid, and recombinant techniques for the production of the polypeptide constructs. Nucleic acids of interest encode a polypeptide that is at least about 80% identical to the provided polypeptide sequences, at least about 85%, at least about 90%, at least about 95%, at least about 99%, or identical. Polynucleotide sequences may encode any or all of the provided sequences, or may encode a fusion protein such as an Fc fusion, a CAR.

In some embodiments, a vector comprising a coding sequence that encodes TRACeR sequence or TRACeR construct is provided, where the coding sequence is operably linked to a promoter active in the desired cell; or is provided in a vector suitable for genomic insertion, e.g., by CRISPR. Various vectors are known in the art and can be used for this purpose, e.g., viral vectors, plasmid vectors, minicircle vectors, which vectors can be integrated into the target cell genome, or can be episomally maintained.

Polypeptide compositions may be prepared as aerosols, injectables, either as liquid solutions or suspensions; solid forms suitable for solution in, or suspension in, liquid vehicles prior to injection can also be prepared. Proteins can be administered in the form of a depot injection or implant preparation which can be formulated in such a manner as to permit a sustained or pulsatile release of the active ingredient. The pharmaceutical compositions are generally formulated as sterile, substantially isotonic and in full compliance with all Good Manufacturing Practice (GMP) regulations of the U.S. Food and Drug Administration.

The preferred form depends on the intended mode of administration and therapeutic application. The compositions can also include, depending on the formulation desired, pharmaceutically-acceptable, non-toxic carriers or diluents, which are defined as vehicles commonly used to formulate pharmaceutical compositions for animal or human administration. The diluent is selected so as not to affect the biological activity of the combination. Examples of such diluents are distilled water, physiological phosphate-buffered saline, Ringer’s solutions, dextrose solution, and Hank’s solution. In addition, the pharmaceutical composition or formulation may also include other carriers, adjuvants, or nontoxic, nontherapeutic, nonimmunogenic stabilizers and the like.

Acceptable carriers, excipients, or stabilizers are non-toxic to recipients at the dosages and concentrations employed, and include buffers such as phosphate, citrate, and other organic acids; antioxidants including ascorbic acid and methionine; preservatives (such as octadecyidimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g., Zn-protein complexes); and/or non-ionic surfactants such as TWEEN™, PLURONICS™ or polyethylene glycol (PEG).

In another embodiment of the invention, an article of manufacture containing an isolated polypeptide or polynucleotide is provided. The article of manufacture comprises a container and a label. Suitable containers include, for example, bottles, vials, syringes, and test tubes. The containers may be formed from a variety of materials such as glass or plastic. The container holds a polypeptide or polynucleotide composition, which may be a therapeutic composition, e.g. for treatment of cancer, and may have a sterile access port (for example the container may be an intravenous solution bag or a vial having a stopper pierceable by a hypodermic injection needle). A label on or associated with the container may indicate that the composition is used for treating the condition of choice. Further container(s) may be provided with the article of manufacture which may hold, for example, a pharmaceutically-acceptable buffer, such as phosphate-buffered saline, Ringer’s solution or dextrose solution. The article of manufacture may further include other materials desirable from a commercial and user standpoint, including other buffers, diluents, filters, needles, syringes, and package inserts with instructions for use.

Engineered cells can be provided in pharmaceutical compositions suitable for therapeutic use, e.g. for human treatment. Therapeutic formulations comprising such cells can be frozen, or prepared for administration with physiologically acceptable carriers, excipients or stabilizers (Remington’s Pharmaceutical Sciences 16th edition, Osol, A. Ed. (1980)), in the form of aqueous solutions. The cells will be formulated, dosed, and administered in a fashion consistent with good medical practice. Factors for consideration in this context include the particular disorder being treated, the particular mammal being treated, the clinical condition of the individual patient, the cause of the disorder, the site of delivery of the agent, the method of administration, the scheduling of administration, and other factors known to medical practitioners.

The cells can be administered by any suitable means, usually parenteral. Parenteral infusions include intramuscular, intravenous (bolus or slow drip), intraarterial, intraperitoneal, intrathecal or subcutaneous administration.

### Methods of Treatment

The invention further provides methods for reducing viral infection. The TRACeR sequence may be conjugated to a drug that reduces cell growth, e.g. chemotherapeutic drug, toxin, etc. Thus, in some embodiments, the invention provides a method of delivering a drug to a cell, comprising administering a drug-TRACeR complex to a subject. Targeting can be accomplished by coupling (e.g., linking, directly or via a linker molecule, either covalently or non-covalently, so as to form a drug-antibody complex) a drug to an antibody specific for a cancer-associated polypeptide. Methods of coupling a drug to a protein are well known in the art.

### Methods of Screening

Compositions and methods are provided for accurately identifying proteins that specifically bind to a peptide in a given MHC context. The methods involve the generation of a diverse library of de novo designed peptides, e.g. comprising TRACeR library polypeptides, which are contacted with specific MHC binding domains, which provide the MHC context, complexed with peptide ligands. The diversity of the library is as previously defined.

The peptide ligand is from about 8 to about 20 amino acids in length, usually from about 8 to about 18 amino acids, from about 8 to about 16 amino acids, from about 8 to about 14 amino acids, from about 8 to about 12 amino acids, from about 10 to about 14 amino acids, from about 10 to about 12 amino acids.

The library can be provided in the form of a polynucleotide, e.g. a coding sequence operably linked to an expression vector; which is introduced by transfection, electroporation, etc. into a suitable host cell. Eukaryotic cells are preferred as a host, and may be any convenient host cell that can be transfected and selected for expression of a protein on the cell surface. Yeast cells are a convenient host, although are not required for practice of the methods.

Once introduced in the host cells, expression of the library is induced and the cells maintained for a period of time sufficient to provide cell surface display of the polypeptides of the library.

Selection for a protein that binds to the MHC-peptide of interest is performed by combining a multimerized MHC with the population of host cells expressing the library. See Overall S.A. et al., and Sgourakis N.G, Nature Communications (2020) and WO 2020/010261 each herein specifically incorporated by reference. The multimerized MHC-peptide for selection is a soluble protein comprising the binding domains of an MHC of interest complexed with a peptide, and can be synthesized by any convenient method. The MHC may be a single chain, or a multimer, dendrimer, etc., and can comprise a detectable label, e.g. a fluorophore, mass label, etc., or can be bound to a particle, e.g. a paramagnetic particle. Selection of cells bound to the MHC-peptide can be performed by flow cytometry, magnetic selection, and the like as known in the art.

Rounds of selection may include one or more rounds of negative selection for binding to non-cognate MHC proteins, and/or to negative selection for binding to non-cognate peptides.

Rounds of selection are performed until the selected population has a signal above background, usually at least three and more usually at least four rounds of selection are performed. In some embodiments, initial rounds of selection, e.g. until there is a signal above background, are performed with an MHC coupled to a magnetic reagent, such as a superparamagnetic microparticle, which may be referred to as “magnetized”. Alternatively, one may also use second stage antibodies that recognize species-specific epitopes of the MHC, e.g. anti-mouse Ig, anti-rat Ig, etc. Indirect coupling methods allow the use of a single magnetically coupled entity, e.g. antibody, avidin, etc., with a variety of separation antibodies.

Alternatively, the MHC is multimerized to a reagent having a detectable label, e.g. for flow cytometry, mass cytometry, etc. For example, FACS sorting can be used to increase the concentration of the cells of having a peptide ligand binding to the TCR. Techniques include fluorescence activated cell sorters, which can have varying degrees of sophistication, such as multiple color channels, low angle and obtuse light scattering detecting channels, impedance channels, etc.

After a final round of selection, polynucleotides are isolated from the selected host cells, and the sequence of the selected binders are determined, usually by high throughput sequencing. The desired affinity may be at less than about 10⁻⁷ kd less than about 10⁻⁸, less than about 10⁻⁹, less than about 10⁻¹⁰, less than about 10⁻¹¹.

The peptide sequence results and database search results may be provided in a variety of media to facilitate their use. “Media” refers to a manufacture that contains the expression repertoire information of the present invention. The databases of the present invention can be recorded on computer readable media, e.g. any medium that can be read and accessed directly by a computer. Such media include, but are not limited to: magnetic storage media, such as floppy discs, hard disc storage medium, and magnetic tape; optical storage media such as CD-ROM; electrical storage media such as RAM and ROM; and hybrids of these categories such as magnetic/optical storage media. One of skill in the art can readily appreciate how any of the presently known computer readable mediums can be used to create a manufacture comprising a recording of the present database information. “Recorded” refers to a process for storing information on computer readable medium, using any such methods as known in the art. Any convenient data storage structure may be chosen, based on the means used to access the stored information. A variety of data processor programs and formats can be used for storage, e.g. word processing text file, database format, etc.

As used herein, “a computer-based system” refers to the hardware means, software means, and data storage means used to analyze the information of the present invention. The minimum hardware of the computer-based systems of the present invention comprises a central processing unit (CPU), input means, output means, and data storage means. A skilled artisan can readily appreciate that any one of the currently available computer-based system are suitable for use in the present invention. The data storage means may comprise any manufacture comprising a recording of the present information as described above, or a memory access means that can access such a manufacture.

A variety of structural formats for the input and output means can be used to input and output the information in the computer-based systems of the present invention. Such presentation provides a skilled artisan with a ranking of similarities and identifies the degree of similarity contained in the test expression repertoire.

### EXAMPLES

The following examples are offered by way of illustration and not by way of limitation.

### Example 1

A novel pMHC binding platform inspired by Mycoplasma arthritidis mitogen (MAM). TCRs are the only known proteins that bind pMHCs at varying affinities according to the sequences of the embedded peptide. Other than TCRs, the only other class of proteins in the Protein Data Bank that has been co-crystalized with MHCs are superantigens, but superantigens bind in the constant regions of MHC and TCR, crosslinking them to achieve T-cell activation regardless of the peptide identity. There is, however, one unique crystal structure of the ternary complex of MAM with TCR and pMHC-II (PDB ID: 2ICW). While most superantigens, such as staphylococcal enterotoxin A, B, and H (SEA, SEB, SEH, etc.) bind pMHC-lls away from the peptide, MAM interacts with pMHC-II via a helical bundle in a position right above the peptide binding groove. The N-terminal residues of the helical bundle also form an unstructured loop, lying above and parallel to the MHC-bound peptide in space. Redesigning the loop can provide peptide selectivity.

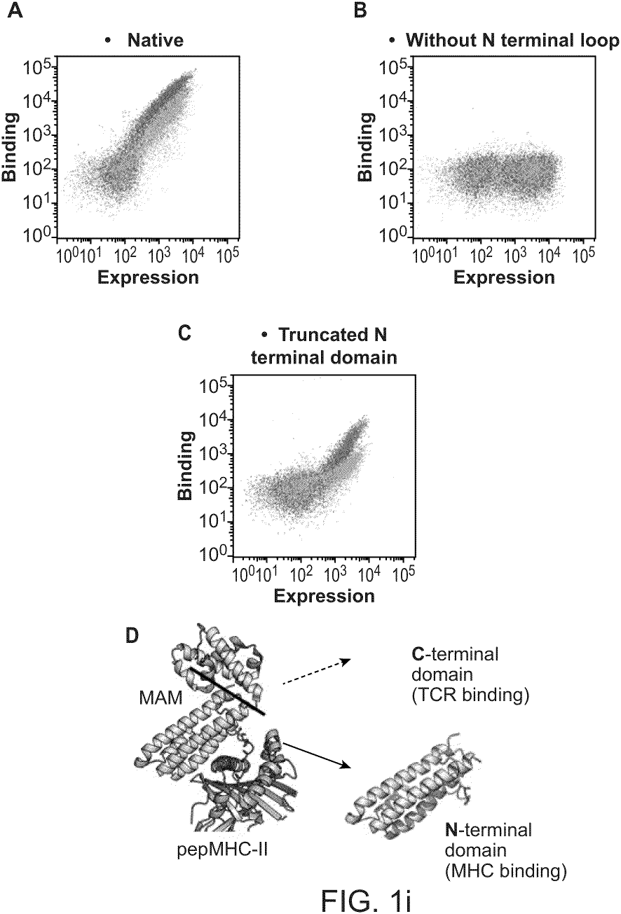

Keeping the MHC interacting domain of MAM and modifying the structure to carry an additional peptide recognition element, the redesigned MAM was able to differentiate two different peptides on the same class-II MHC. The redesigned MAM was expressed on the yeast surface and probed with soluble pMHC with either the influenza hemagglutinin peptide (HA) or the class II-associated invariant chain peptide (CLIP). The same MHC-II, HLA-DR1, was used with the two different peptides, so the difference in binding is solely due to the peptide identities. Using flow cytometry, we found that a library designed for HA with 10⁵ variants contains binders that bind HA-loaded pMHC-II at high affinity, while rejecting CLIP pMHC-II completely. (see FIGS. 1A-1E). This evidence demonstrates that a modified superantigen can be used to read the identity of the MHC peptide.

Using pMHC library to screen for target antigens. We use a breakthrough method for generating class-I pMHC libraries carrying peptides of interest. The unique capability of this technology is that it can generate individually DNA-barcoded and fluorescently-labelled pMHCs in a high-throughput fashion, e.g. the loading of a dominant NY-ESO-1 peptide on the class-I MHC, HLA A*02:01, which is an allotype common in humans. This platform was used to select for positive binders against NY-ESO-1 antigens.

The general platform is used for a variety of other applications, including payload delivery, imaging and further mapping and characterization of the SARS-CoV-2 epitopes on cell surfaces. The strategy to develop antigen-peptide MHC binders is applied to other viral infections and cancer.

### Example 2

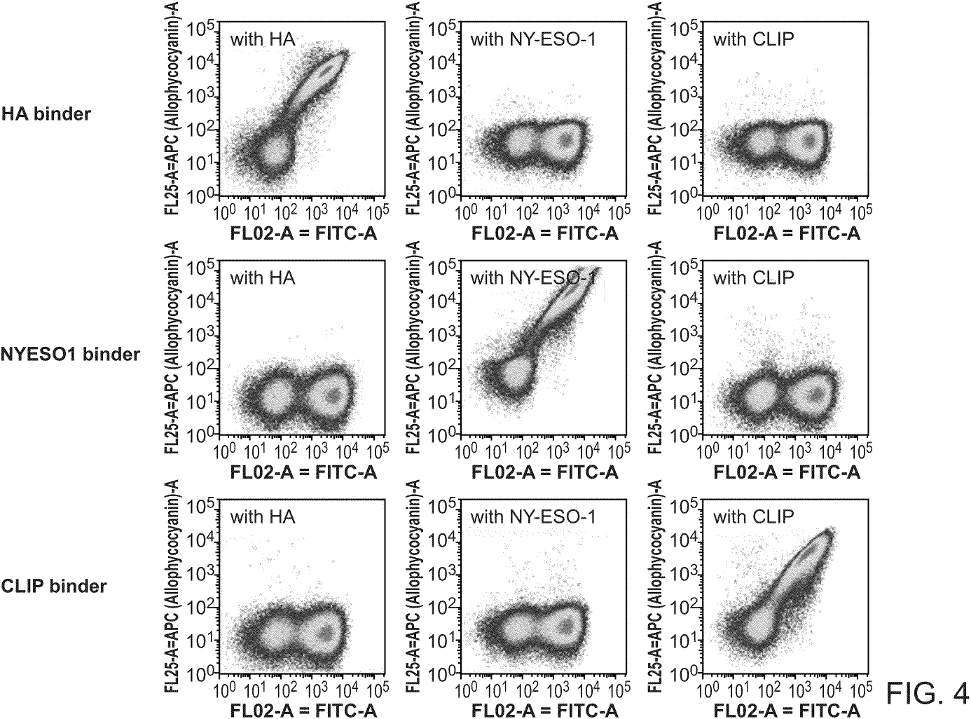

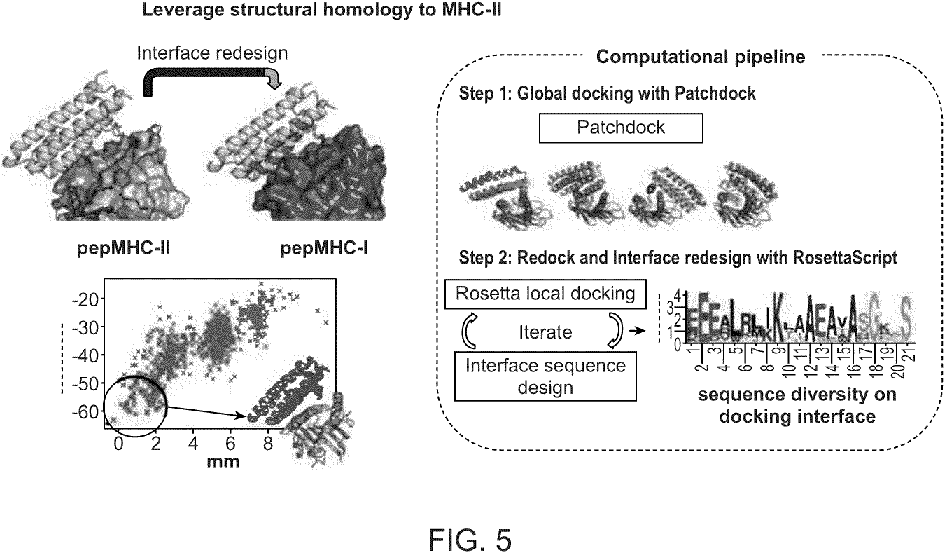

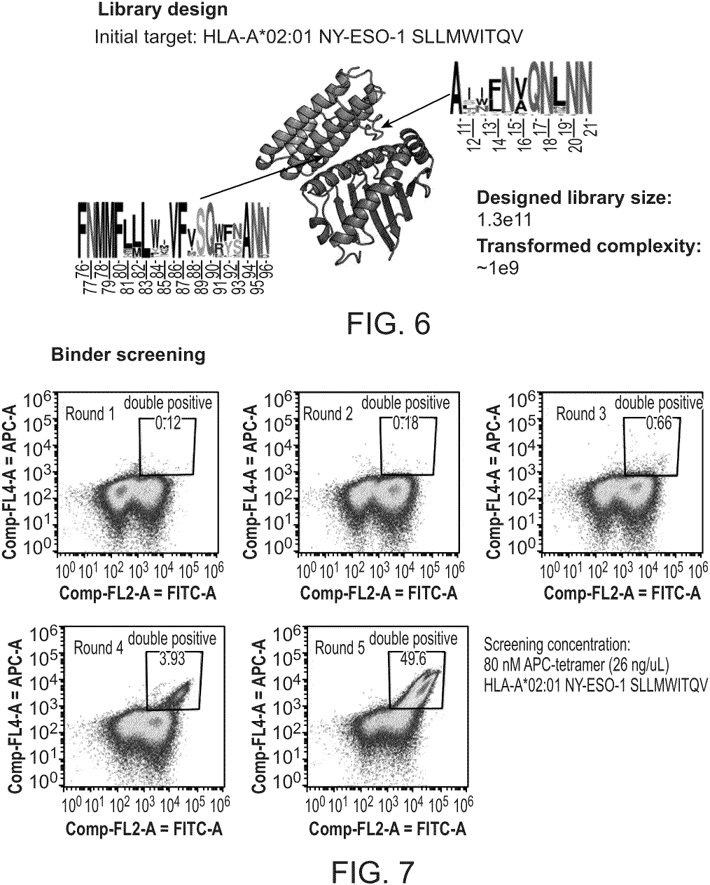

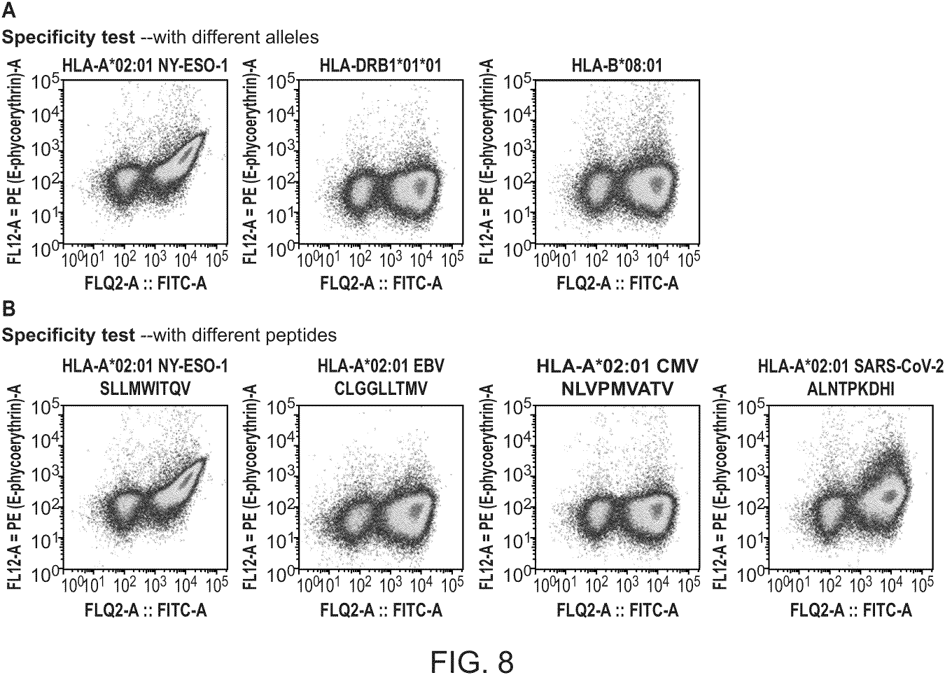

Computational design of MHC-I antigen-specific binders. Starting with the designed model, which consists of a four-helix bundle (N-terminal domain of MAM) and an additional helix we designed to anchor in the MHC peptide groove, for MHC-I binding. We defined new binding poses between the binder and MHC before further redesigning the sequences at the interface. For example, dominant pMHC-I antigens of the entire SARS-CoV-2 sequence have been computationally identified for HLA A*02:01. We use the 5 most stable pMHC-I models to design the pMHC-I binder, for example as shown in FIGS. 5, 6, 7 and 8.

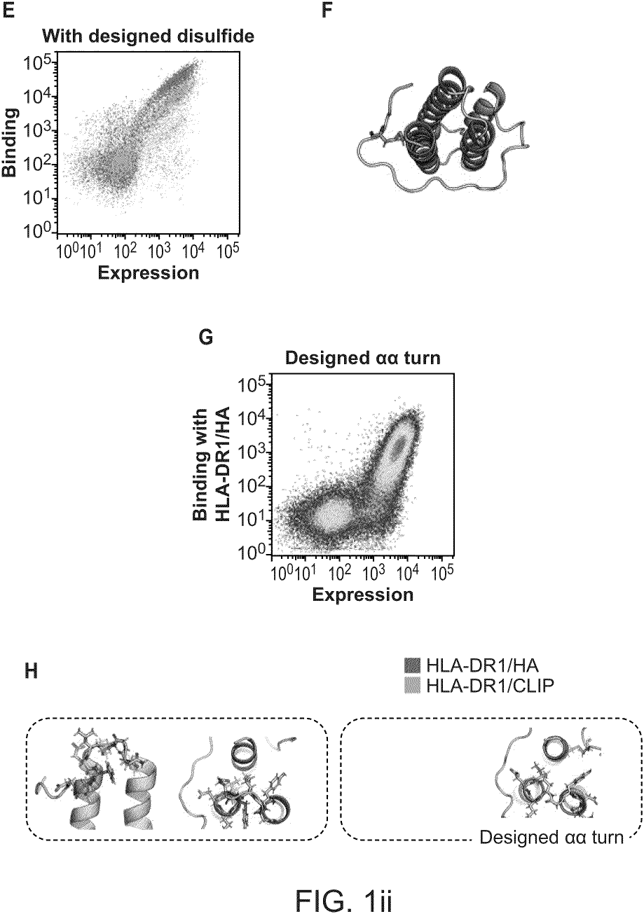

PatchDock correlates the convex and concave surfaces between proteins, to coarsely dock the two structures together according to their shape complementarity. A Rosetta protocol previously developed for designing a flu virus inhibitor on the top models from PatchDock to refine the docked poses, as well as redesigning the amino acids according to the Rosetta energy function. The Rosetta protein-protein interface design protocol will carry out iteratively (i) rigid-body docking, (ii) sequence design, and (iii) backbone refinement steps. (FIG. 2) The resulting models are analyzed by their computed binding energy, defined as the difference in energy between the unbound and the bound state (ddG) of the binding partners. We manually analyze the top scoring models, as well as using a variety of metrics, such as hydrogen-bonding satisfaction and sidechain packing density, to evaluate the designs. We compile the mutations suggested by Rosetta either into a focused combinatorial library or as individual constructs for experimental characterization.

A critical consideration for the docked poses is the location of the peptide recognition element pMHC-l peptides are usually 8-10 residues in length with both ends of the peptide anchored into the binding groove. Because of the conserved anchoring interactions with the MHC, the structural differences between different peptides lie mostly within the central 4-5 residues of the peptide. The peptide recognition element needs to have meaningful contacts with this region. If the docking and design procedure is unable to locate the recognition element or produce proper sequences to interact with the peptide, we repeat the process on different designs of the peptide recognition element. We started out designing it as a simple helix, but other structures, such as loops, beta-hairpins, or helical hairpins could alternatively be used.

Experimental optimization of designed NY-ESO-1 pMHC-I binders. We tested combinatorial libraries of 10⁷ complexity on yeast surface display. Constructs were probed with fluorescently labeled NY-ESO-1 peptide-loaded MHC-I using fluorescence-activated cell sorting (FACS). Sequences (or libraries) designed for a specific peptide were first selected with the cognate peptide, then checked (and negatively selected) against the other non-NY-ESO-1 HLA-A02 antigens to assess cross-reactivity. Multiple rounds of optimization were conducted.

### Example 3

COVID-19 symptoms are the results of the innate immune response attempting to manage viral spread, but failing to match the rate of viral replication. The progression of disease is usually not controlled until the adaptive immune response can mount efficient antibody countermeasures. In general, the ability of the initial cellular response to control the rate of viral spread is a race against numbers and time: the rate at which viruses are produced from cells and the chances the immune surveillance can effectively detect and eradicate diseased cells. For patients who suffer severe symptoms, the immune system falls behind, and one of the main reasons that the immune system is at a disadvantage is that it takes time to match among the large TCR pool for the specific antigens the immune system has never seen before. Even with approximately 10¹¹ T cells in circulation, along with further expansion triggered by inflammation, it is still difficult for cellular immunity to maintain control for viruses as potent as SARS-CoV-2.

Current antiviral strategies largely depend on small-molecule drugs that target various viral replication machineries, such as the Angiotensin-converting enzyme II (ACE2) entry receptor (e.g. chloroquine, hydroxychloroquine), the RNA-dependent RNA polymerase (RdRp) (e.g. remdesivir, favipiravir) and the protease proteins (e.g. disulfiram, lopinavir), focusing on blocking penetration or interfering with viral replication. However, since the modality of administering the drugs is systemic, earlier studies have indicated significant side effects for ACE2 inhibitors and protease inhibitors, and administration dosage has to be controlled at a low level. Thus, even for small-molecule drugs, having a targeted delivery system can be highly beneficial.

There is a critical need for new methods to minimize community spread of COVID19. The COVID19 coronavirus is highly contagious, and aerosolized viral particles released by infected cells of the respiratory pathways have been shown to readily contaminate exposed surfaces on objects or skin; and mucous membranes in the eyes, nose, and oral surfaces of individuals within close proximity of an infected person. COVID19 viral particles have been shown to retain their infectivity on surfaces for as long as 2 weeks and up to 48 hours suspended in air as aerosolized particles.

The COVID-19 pandemic is a public health emergency caused by the Severe Acute Respiratory Syndrome coronavirus-2 ( SARS-CoV-2). Functional mimetics of T cell receptors that are highly specific for SARS-CoV-2 antigens on Major Histocompatibility Complex-I (MHC-I) and use the designed proteins to target and destroy virally infected cells. By engineering tools that leverage immune mechanisms without susceptibility to viral immune evasion, the strategy is transformative in treating early onset and advanced COVID-19 disease.

We detect virally infected cells as a strategy to treat COVID-19 and other viral infections by designing functional mimetics of T cell receptors (TCRs) that are highly specific for viral antigens presented by MHC-I and develop them into therapeutic agents by making fusion to the Fc-receptor as a novel antiviral treatment. Making fusions with Fc is a strategy that has been used in cancer immunotherapy to induce antibody dependent cellular cytotoxicity (ADCC). We create highly specific proteins recognizing viral peptides presented on MHC-I and that can be used to target/kill infected cells at an early stage. By eradicating infected cells we can limit viral replication and release, mitigate tissue damage and eradicate the virus.

TCRs have evolved to recognize the peptide identities on MHC/peptide complexes (pMHCs). However, TCR engineering remains a challenge. Here we create a non-TCR protein platform to recognize pMHCs. Building on a stable fragment of a native protein, the novel scaffold simplifies the process of creating specific pMHC binders, as the designs will no longer require the involvement of six variable loops like TCRs. Computational protein design methods are combined with experimental high throughput screen techniques.

Specifically, we (1) engineer SARS-CoV-2 MHC antigen-specific binders, (2) ensure their safety by screening their binding strength to minimize cross-reactivity with self-MHC-peptides, and (3) generating therapeutic agents, e.g. pMHC-Fc receptor fusions, to recognize and eliminate SARS-CoV2 infected cells.

This work provides a new platform of novel peptide-specific pMHC binders with broad applicability in biomedical research. Targeting diseased cells by reading their specific pMHC is of great significance for delivering antiviral drugs or inducing immune responses to infection (with strategies such as Fc fusion). As a one-of-a-kind epitope-specific targeting system, this strategy holds promise in treating SARS-CoV-2 and other viral infections.

Developing a general platform for targeting infected cells is broadly beneficial for treating viral infection diseases. Cells present internal proteins on their major histocompatibility complexes (MHCs); class-I MHCs present self-peptides and class-II MHCs are associated with antigen presenting cells to augment the immune response. As virally infected host cells are hijacked to produce viruses, the viral proteins can also be presented on class-I MHCs. It is thus highly desirable to potentially use the peptide-loaded MHCs (pMHCs) for identifying cells that are infected by viruses. Nature uses T-cell receptors (TCRs) to interact with MHCs to identify the presence of foreign antigens among self-peptides. However, the engineering of antigen-specific T cell receptors (TCRs) remains a challenge. Leading technologies for engineering TCRs use mutagenic libraries or TCRs′ natural genetic repertoire to discover TCRs that respond to specific pMHCs. Due to the unique docking geometry required, as well as central tolerance imposing restrictions on TCR affinity, engineered TCRs have been shown to be limited in their ability to achieve optimal binding affinity or avoid cross-reactivity. In general, we still lack broadly applicable technologies that can mimic the immune system and identify cells affected by infection or malignant transformation. Providing specific viral pMHC targeting is transformative for our ability to detect and control the spread of viruses inside the body.

Provided is a novel protein-recognition platform engineered to detect specific antigen epitopes presented in the context of MHC-I. The binding mechanism is different from that of a TCR. Instead of using six distinct structural loops, our design localizes antigen recognition to a single structural element that runs parallel to the antigen binding groove. By designing highly specific “readers’ for pMHC-I presenting viral antigens, we provide a general therapeutic strategy for treating COVID-19 and other viral infections.

### Example 4

### Evaluating Cross-Reactivity With Self-Peptides In Vitro

Using primary thymic tissues epithelial cells to identify possible off-target cross-reactivity with self-peptides. Thymic tissues containing cortical and medullary thymic epithelial cells (cTECs and mTECs) are responsible for the positive and negative selection of developing T cells in the thymus. The peptide repertoire presented on primary thymic epithelial cells may be used to assess the binding affinity of MHC binders to self-peptides and to eliminate those MHC binders that adhere to primary TECs. Primary thymic tissues become available when children with congenital heart defect undergo corrective surgery, and pieces of the thymus need to be removed. A protocol for the dissociation of thymic tissue and enrichment of TECs based on negative selection of CD45 positive T cells, followed by positive selection of EPCAM positive TECs can be used. Green fluorescent protein (GFP) fusions for each of the candidates produced are made. For the small quantities required for labeling, we express these constructs in a cell-free expression system (e.g. PURExpress system from Qiagen), so we can sufficiently screen a reasonable number of constructs (in multiples of 96 plate wells). The primary human thymic tissues are dissociated into suspension and mixed with the GFP-tagged binders in different concentrations for flow cytometry or FACS analysis. We can produce a single-chain version of a TCR (TCR A6) known to react with self-peptides on HLA A*02:01 as the positive control. Binding signals to the TECs can reveal whether the positively enriched pMHC-I binders have the potential to cross-react with self-peptides.

By contrasting the signals from the positive and negative controls, we identify and eliminate the constructs that have the potential to cross-react with self-peptides on pMHC. Because of the large number of constructs we will screen, we expect that there will be highly specific binders with no cross-reactivity. If this is not the case, since a subset of the binders will be derived from combinatorial libraries and from multiple rounds of optimization, we can expand to include more sequences from the set. If the TECs cannot unambiguously differentiate binding events from background signals, we can alternatively use the MHC library platform, loading with known self-peptides for HLA A*02:01, to restrict the cross-reactivity screen to a smaller subset of self-peptides. MHC tetramers containing the positive and negative targets are used directly to optimize constructs in a high-throughput fashion. By incorporating (a smaller range of) negative selection targets, constructs derived from this modified pipeline may gain better signal-to-noise for the TECs. We will then repeat the thymic tissue test with these candidates.

### Example 5

### Testing Immunogenicity in Mice

Testing binder Immunogenicity in mice. Using only the N-terminal domain of MAM is insufficient for the construct to function as a superantigen. We test at least 15 different constructs designed for the 5 different SARS-CoV-2 peptides (3 each), using HEK293e cells for expression. Polyclonal antibodies for ELISA positive control are generated by vaccinating mice with N-terminal domain of MAM in complete Freund’s adjuvant. The antibody titers are characterized by ELISA with absorbance; power calculation (σ=0.3, | µ - µ ∘|=0.5) suggests that a sample size of n>=4 will yield statistical power of greater than 0.8. We use 5 C57BL/6 mice (mixed sexes, 6-8 weeks old) for each construct in the immunogenicity tests. A daily 10 µg purified protein dose is delivered by intraperitoneal injection for 14 days before serum is collected for ELISA analysis. ELISA targets include protein constructs, N-terminal domain of MAM, and ovalbumin (negative protein control).

Native superantigens are highly immunogenic because they directly engage immunological synapses, but the N-terminal domain of MAM used in these designs does not form the synapse; it lacks the C-terminal domain to engage TCR. Should undesirable response arise, we will explore alternative sequences by redesigning the molecules without perturbing the structure. We can also build the helical bundles completely de novo and erase any resemblance to MAM. In regards to potential B-cell epitopes, we can PEGylate the constructs, as well as computationally designing glycan sites on the proteins to mask epitopes, to reduce immunogenicity. Most small, stable, designed proteins tested to date show few immunogenicity issues because of their relatively simple composition.

### Example 6

### Implementing Designed SARS-CoV-2 Antigen Binders as Functional Biologics

Epitope-specific binders are turned into antibody mimetics for therapeutic application. For example, the designed binders are linked to various payloads or immunoglobulin (Ig) domains, following the successful examples of therapeutic antibody drug conjugates, monoclonal antibodies, knottins, and soluble TCRs. The binders are built and tested as IgFc fusions, using the Fc domain to trigger ADCC or phagocytosis. In vitro assays are used to test the cytotoxicity on tissue cultures transfected to express the a selected antigenic peptide.

Building and testing fusions of pMHC-specific binders with IgFc. Fusion constructs are expressed in HEK293e cells as HIS-tagged secreted proteins. The proteins are purified, and the dimerization of the Fc domains is assessed by SDS-PAGE or size-exclusion chromatography. Separate cell lines (as target cells) are created, expressing GFPs and individually the 5 dominant SARS-CoV-2 peptides for MHC presentation on cell surfaces. We will mix 10 × 10⁶ target cells with varying concentrations of the Fc fusion constructs and the controls, and use ADCC reporter cell lines (e.g. Jurkat-Lucia-NFAT-CD16 cells) to measure the level of ADCC induced by the Fc fusion constructs. A number of similar commercially available reporter cell lines give luciferase readouts. The Fc fusion constructs are tested as singles, doubles, or multiplexes (but normalized by total fusion concentration). Normalized cell deaths mediated by the Fc fusion constructs reveal the efficacy of function candidates, as well as their effective tagging of the respective antigen peptides.

The Fc fusion constructs are expected to deliver cytotoxicity to the target cells. Alternatively constructs are drug conjugates, or fusions with potent cytotoxins such as Pseudomonas exotoxin A, to deliver targeted cytotoxicity. Antiviral drugs are conjugated via click chemistry by attaching an azide tag on the drug and using an cyclooctyne-NHS linker (e.g. DBCO-NHS) to conjugate with the protein. If direct conjugation of protein and drug cannot work, drugs can be encapsulated in biodegradable polymeric nanocapsules (e.g. PEG-PLGA) and the nanoparticles conjugated to proteins.

### Example 7

### Recognizing Cancer-Related Antigen

A novel protein engineering platform, designed to target pMHC-I complexes that present specifically targeted epitopes is provided. The provided design localizes antigen recognition to a single structural element that runs parallel to the antigen binding groove. Pair-wise screening against cross-reactivity with self-peptides can be deployed. By designing highly specific “readers” for pMHC-I presenting viral antigens, a general therapeutic strategy is provided for treating cancer, autoimmunity, infectious-disease and serious chronic inflammatory conditions. The therapeutic molecule is selected from a TRACeR library as described herein. This targeting strategy is augmented with effector-functionalized fusion-proteins for direct killing of pathogenic cells, or to recruit and activate cytotoxic T-cells, antibody-dependent cellular cytotoxicity (ADCC) or phagocytosis.

Engineer MHC neoantigen-specific binders based on a concept inspired by Mycoplasma arthritidis mitogen (MAM). A TRACeR library for MHC Class I/peptide complex screening is screened for binding to an MHC/cancer peptide complex of interest. These experiments produce peptide-specific pMHC-I binders for initially targeted cancer-targets, e.g. NY-ESO-1 and ALK.

The selected TRACeR binding protein is fused to an anti-CD3 scFv (or Fc or other effector “tagged”) fusions as a strategy to recruit activated T-cell populations to the diseased cells or otherwise target the cells displaying the targeted pMHC complex. In vitro assays testing for cytotoxicity are performed. For Fc-fusion constructs, an antibody-dependent cellular cytotoxicity (ADCC) reporter cell line is used, or the construct is introduced into peripheral blood mononuclear cells (PBMCs) to track the efficacy of designs. These experiments determine the cellular-level specificity of designed constructs and establish their potential as protein therapeutics.

### Example 8

The selection process for TRACeR-I (TRACeR targeting class-I MHC) was done in two different stages. The first stage selected for affinity to the MHC-I protein scaffold, e.g. using the library according to SEQ ID NO:5, and the second stage selected for contacts in the antigen binding groove, e.g. using the library of SEQ ID NO:6.

Based on computational models, we identified all the positions involved in the TRACeR-MHC interface and group them into MHC-I contacting or peptide antigen contacting residues. We hope to capture the same binding modes as the computational models experimentally, and thus the sequences considered in the libraries were based on the computational design. The computational design process generated docked ensembles of TRACeR on the MHC target, and the sequences at the interface were designed for each docked pose. From the entire ensemble of a binding mode, we first used the computational energies to collect top ranking structures. For each residue position involved in the binding interface, the identities of allowed amino acids were collected and we used SwiftLib web server to identify the most suitable randomized codon for the position. To reduce the library complexity, the randomized codons for some positions were manually chosen.

The C-terminal portion of the native MAM was truncated, using only used the N-terminal helical bundle structure. The helical bundle structure can also be further modified or be generated de novo. For one of the binding modes for MHC-I, we have to rebuild the structure to avoid hitting a neighboring structural element.

The experimental selection process starts with a first library that randomizes positions involved in MHC-I scaffold contacts (SEQ ID NO:5). This is to ensure that the TRACeR designs have basal affinities for MHC-I before introducing antigen specificity. The positions carrying randomized codons were scattered throughout the sequence, built with oligomers designed to make sure that the randomized codon positions are not involved in the oligo overlapping regions. The oligo overlaps are optimized by the program PRIDE (written by Possu Huang) to all contain 10 to 11 GCs out of 22 bases.

We used binding sequences from the first screen and added randomized codons corresponding to the positions on the peptide-recognition element to make a second library (SEQ ID NO:6). The different sequences from the first library screen can built into different libraries in the second screen or they can be mixed into one single library.

TRACeR library was displayed on yeast and the binding to MHC-I carried out with fluorescently labelled MHC-tetramers, although MHC-dextramers or MHC-monomers can be used, all carrying the selected target peptide. We conducted multiple rounds of library enrichment and sorting on FACS to create antigen specific TRACeRs.

### Example 9

### Sequence Examples

A pilot library of an MHC Class II library has the sequence formula of SEQ ID NO:3:

MKLRCENPKKAXXXXXQNLNNVVFTNKELEDIYDLSNKEETKEVLKLFKL
KVNQFYRHAFGIVNDYNGLLEYKEIFNMMFLKLSVVFDTQRKEANNVEQI
KRNIAILDEIMAKADNDLCYFISQ

(where a “X” denotes any amino acid, as encoded by an NNK randomized codon position).

Selected binders had the following defined PRE sequences (SEQ ID NO:3, residues 12-16), peptide antigens as indicated:

For HA specific binding:

| | | | | | |
|---|---|---|---|---|---|
| E | V | H | M | V | SEQ ID NO:17 |
| D | N | H | L | V | SEQ ID NO:18 |
| E | T | H | W | V | SEQ ID NO:19 |
| E | S | H | L | V | SEQ ID NO:20 |
| E | R | H | L | L | SEQ ID NO:21 |
| D | N | H | L | V | SEQ ID NO:22 |
| E | V | H | W | V | SEQ ID NO:23 |
| D | W | H | L | V | SEQ ID NO:24 |

For NYESO-1 specific binding:

| | | | | | |
|---|---|---|---|---|---|
| F | R | W | G | G | SEQ ID NO:25 |
| V | R | W | G | G | SEQ ID NO:26 |
| V | R | W | G | G | SEQ ID NO:27 |
| V | K | W | G | G | SEQ ID NO:28 |
| Y | V | L | G | V | SEQ ID NO:29 |
| G | R | F | G | V | SEQ ID NO:30 |
| Y | R | W | G | G | SEQ ID NO:31 |
| V | R | W | G | G | SEQ ID NO:32 |

For CLIP specific binding:

| | | | | | |
|---|---|---|---|---|---|
| S | T | Y | L | A | SEQ ID NO:33 |
| S | I | F | L | A | SEQ ID NO:34 |
| Q | L | F | L | A | SEQ ID NO:35 |
| V | V | F | L | V | SEQ ID NO:36 |
| V | L | F | L | V | SEQ ID NO:37 |
| A | V | Y | L | A | SEQ ID NO:38 |
| A | V | Y | G | A | SEQ ID NO:39 |
| T | A | Y | G | A | SEQ ID NO:40 |

The peptides screened were SEQ ID NO:7 HLA-DR1/HA (PKYVKQNTLKLAT); SEQ ID NO:8 HLA-DR1/ NY-ESO-1(LLEFYLAMPFATPME); SEQ ID NO:9 HLA-DR1/CLIP (PVSKMRMATPLLMQA).

Exemplary, individual, specific binding proteins in an HLA-DR1 context were as follows. Variable sequences are in bold. For HA antigen, SEQ ID NO:10

MKLRCENPKKAERHLLQNLNNVVFTNKELEDIYDLSNKEETKEVLKLFKL
KVNQFYRHAFGIVNDYNGLLEYKEIFNMMFLKLSVVFDTQRKEANNVEQI
KRNIAILDEIMAKADNDLCYFISQ

For NY-ESO-1 antigen, SEQ ID NO:11

MKLRCENPKKAYVLGVQNLNNVVFTNKELEDIYDLSNKEETKEVLKLFKL
KVNQFYRHAFGIVNDYNGLLEYKEIFNMMFLKLSVVFDTQRKEANNVEQI
KRNIAILDEIMAKADNDLCYFISQ.

and SEQ ID NO:12

MKLRCENPKKAVRWGGQNLNNVVFTNKELEDIYDLSNKEETKEVLKLFKL
KVNQFYRHAFGIVNDYNGLLEYKEIFNMMFLKLSVVFDTQRKEANNVEQI
KRNIAILDEIMAKADNDLCYFISQ.

For CLIP antigen, SEQ ID NO:13

MKLRCENPKKAQLFLAQNLNNVVFTNKELEDIYDLSNKEETKEVLKLFKL
KVNQFYRHAFGIVNDYNGLLEYKEIFNMMFLKLSVVFDTQRKEANNVEQI
KRNIAILDEIMAKADNDLCYFISQ.

In library 2, 8 randomized codon positions were introduced, using the sequence formula of SEQ ID NO:4

MKLRCENPKKAXXXXXXXXNNVVFTNKELEDIYDLSNKEETKEVLKLFKL
KVNQFYRHAFGIVNDYGDKEIFNMMFLKLSVVFDTQRKEANNVEQIKRNI
AILDEIMAKADNDLCYFISQ

(where a “X” denotes any amino acid, as encoded by an NNK randomized codon position).

The MHC Class II interface was redesigned to bind to a Class I protein, starting with the formula of SEQ ID NO:5

MKLRCENPKKAXXXNXQNLNNVVFTNKELEDIYDLSNKEETKEVLKLFKL
KVNQFYRHAFGIVNDYGDKEIFNMMFXXLWXVFXSQXXXANNVEXIKXNI
XXLDWIMAEADNDLCYFISQ

The polynucleotide coding sequence used was SEQ ID NO:14:

ATGAAGCTGCGTTGTGAAAATCCGAAGAAAGCCNNKNNKNNKAACSYACA
AAACCTAAATAACGTCGTCTTCACCAACAAAGAGCTTGAAGACATCTATG
ATCTGTCAAATAAGGAAGAGACAAAGGAAGTCCTTAAGCTGTTCAAACTT
AAAGTGAACCAGTTCTATAGACATGCTTTCGGTATTGTCAATGACTATGG
AGATAAGGAGATCTTTAACATGATGTTCVHSMTGCTATGGVNSGTGTTCN
KCAGTCAGARAHDCRVCGCCAACAACGTAGAGSTAATTAAATTMAACATC
ARAVTGTTAGATTGGATAATGGCGGAAGCAGACAATGACTTATGTTACTT
CATTAGTCAA

This library was screened against HLA-A*02:01/NY-ESO-1 SLLMWITQV (SEQ ID NO:15) for binders, providing an binder against HLA-A*02:01/NY-ESO-1 sequence, SEQ ID NO:16:

MKLRCENPKKAYRDNAQNLNNVVFTNKELEDIYDLSNKEETKEVLKLFKL
KVNQFYRHAFGIVNDYGDKEIFNMMFMLLWRVFRSQRIDANNVELIKFNI
RVLDWIMAEADNDLCYFISQ

The sequence formula to create a library on the PRE loop region, for selecting binders for multiple targets in an HLA Class I context, is provided as SEQ ID NO:6

MKLRCENPKXXXXXXXQNLNNVVFTNKELEDIYDLSNKEETKEVLKLFKL
KVNQFYRHAFGIVNDYGDKEIFNMMFMLLWRVFRSQRIDANNVELIKFNI
RVLDWIMAEADNDLCYFISQ.

### BIBLIOGRAPHY

1. Sompayrac, L, M. How the immune system works. John Wiley& Sons . (2019)

2. Schmitt, T. M. et al. Generation of higher affinity T cell receptors by antigen-driven differentiation of progenitor T cells in vitro. Nat. Biotechnol. 35, 1188-1195 (2017).

3. Cao, X. COVID-19: immunopathology and its implications for therapy. Nat. Rev. Immunol. 20, 269-270 (2020).

4. Hancioglu, B., Swigon, D. & Clermont, G. A dynamical model of human immune response to influenza A virus infection. J. Theor. Biol. 246, 70-86 (2007).

5. Huseby, E. S. et al. How the T cell repertoire becomes peptide and MHC specific. Cell 122, 247-260 (2005).

6. Krogsgaard, M. et al. Evidence that structural rearrangements and/or flexibility during TCR binding can contribute to T cell activation. Mol. Cell 12, 1367-1378 (2003).

7. Wu, L. C., Tuot, D. S., Lyons, D. S., Christopher Garcia, K. & Davis, M. M. Two-step binding mechanism for T-cell receptor recognition of peptide MHC. Nature 418, 552-556 (2002).

8. Lythe, G., Callard, R. E., Hoare, R. L. & Molina-París, C. How many TCR clonotypes does a body maintain? J. Theor. Biol. 389, 214-224 (2016).

9. Jakhar, D. & Kaur, I. Potential of chloroquine and hydroxychloroquine to treat COVID-19 causes fears of shortages among people with systemic lupus erythematosus. Nat. Med. 26, 632 (2020).

10. Gautret, P. et al. Hydroxychloroquine and azithromycin as a treatment of COVID-19: results of an open-label non-randomized clinical trial. Int. J. Antimicrob. Agents, 105949 (2020).

11. Li, G. & De Clercq, E. Therapeutic options for the 2019 novel coronavirus (2019-nCoV). Nat. Rev. Drug Discov. 19, 149-150 (2020).

12. Grein, J. et al. Compassionate Use of Remdesivir for Patients with Severe Covid-19. N. Engl. J. Med. 382, 2327-2336 (2020).

13. Sang, P., Tian, S., Meng, Z. & Yang, L. Insight Derived from Molecular Docking and Molecular Dynamics Simulations into the Binding Interactions Between HIV-1 Protease Inhibitors and SARS-CoV-2 3CLpro. ChemRxiv doi: 10.26434/chemrxiv.11932995.v1 (2020) .

14. Mandel, E. H. The Side-effects of chloroquine and hydroxychloroquine: results of a comparative study in vivo. N. Y. State J. Med. 63, 3111-3113 (1963).

15. Huff, A. Protease inhibitor side effects take people by surprise. GMHC Treat. Issues 12, 25-27 (1997).

16. Huang, J., Song, W., Huang, H. & Sun, Q. Pharmacological Therapeutics Targeting RNA-Dependent RNA Polymerase, Proteinase and Spike Protein: From Mechanistic Studies to Clinical Trials for COVID-19. J. Clin. Med. Res. 9, 1131 (2020).

17. Richman, S. A. & Kranz, D. M. Display, engineering, and applications of antigen-specific T cell receptors. Biomol. Eng. 24, 361-373 (2007).

18. Molloy, P. E., Sewell, A. K. & Jakobsen, B. K. Soluble T cell receptors: novel immunotherapies. Curr. Opin. Pharmacol. 5, 438-443 (2005).

19. Krogsgaard, M. & Davis, M. M. How T cells ‘see’ antigen. Nat. Immunol. 6, 239-245 (2005).

20. Li, Y. et al. Directed evolution of human T-cell receptors with picomolar affinities by phage display. Nat. Biotechnol. 23, 349-354 (2005).

21. Fraser, J. D. & Proft, T. The bacterial superantigen and superantigen-like proteins. Immunol. Rev. 225, 226-243 (2008).

22. Wang, L. et al. Crystal structure of a complete ternary complex of TCR, superantigen and peptide-MHC. Nat. Struct. Mol. Biol. 14, 169-171 (2007).

23. Petersson, K., Thunnissen, M., Forsberg, G. & Walse, B. Crystal structure of the D227A variant of Staphylococcal enterotoxin A in complex with human MHC class II. RCSB Protein Data Bank doi: 10.2210/pdb1lo5/pdb (2002) .

24. Jardetzky, T. S. et al. Complex of the human MHC class II glycoprotein HLA-DR1 and the Bacterial superantigen SEB. RCSB Protein Data Bank doi: 10.2210/pdb1seb/pdb (1996) .

25. Sharma, P., Harris, D. T., Stone, J. D. & Kranz, D. M. T-cell Receptors Engineered De Novo for Peptide Specificity Can Mediate Optimal T-cell Activity without Self Cross-Reactivity. Cancer Immunol. Res. 7, 2025-2035 (2019).

26. Arstila, T. P. et al. A direct estimate of the human alphabeta T cell receptor diversity. Science 286, 958-961 (1999).

27. Goldrath, A. W. & Bevan, M. J. Selecting and maintaining a diverse T-cell repertoire. Nature 402, 255-262 (1999).

28. Anderson, G. & Takahama, Y. Thymic epithelial cells: working class heroes for T cell development and repertoire selection. Trends Immunol. 33, 256-263 (2012).

29. Kondo, K., Takada, K. & Takahama, Y. Antigen processing and presentation in the thymus: implications for T cell repertoire selection. Curr. Opin. Immunol. 46, 53-57 (2017).

30. Murata, S. et al. Regulation of CD8+ T cell development by thymus-specific proteasomes. Science 316, 1349-1353 (2007).

31. Takada, K. et al. TCR affinity for thymoproteasome-dependent positively selecting peptides conditions antigen responsiveness in CD8(+) T cells. Nat. Immunol. 16, 1069-1076 (2015).

32. Nitta, T. et al. Thymoproteasome shapes immunocompetent repertoire of CD8+ T cells. Immunity 32, 29-40 (2010).

33. Overall, S. A. et al. High throughput pMHC-I tetramer library production using chaperone mediated peptide exchange. Nat. Commun. 11, 1909 (2020).

34. Mosquera, L. A. et al. In vitro and in vivo characterization of a novel antibody-like single-chain TCR human IgG1 fusion protein. J. Immunol. 174, 4381-4388 (2005).

35. Nerli, S. & Sgourakis, N. G. Structure-based modeling of SARS-CoV-2 peptide/HLA-A02 antigens bioRxiv (2020) doi: 10.1101/2020.03.23.004176 .

36. Schneidman-Duhovny, D., Inbar, Y., Nussinov, R. & Wolfson, H. J. PatchDock and SymmDock: servers for rigid and symmetric docking. Nucleic Acids Res. 33, W363-W367 (2005).

37. Fleishman, S. J. et al. Computational design of proteins targeting the conserved stem region of influenza hemagglutinin. Science 332, 816-821 (2011).

38. Fleishman, S. J. et al. RosettaScripts: a scripting language interface to the Rosetta macromolecular modeling suite. PLoS One 6, e20161 (2011).

39. Borbulevych, O. Y., Piepenbrink, K. H. & Baker, B. M. Conformational melding permits a conserved binding geometry in TCR recognition of foreign and self molecular mimics. J. Immunol. 186, 2950-2958 (2011).

40. Smith, S. N. et al. Changing the peptide specificity of a human T-cell receptor by directed evolution. Nat. Commun. 5, 5223 (2014).

41. Zhu, X. et al. Visualization of p53264-272/HLA-A*0201 complexes naturally presented on tumor cell surface by a multimeric soluble single-chain T cell receptor. J. Immunol. 177, 5747 (2006).

42. Silva, D.-A. et al. De novo design of potent and selective mimics of IL-2 and IL-15. Nature 565, 186-191 (2019).

43. Huang, P.-S. et al. High thermodynamic stability of parametrically designed helical bundles. Science 346, 481-485 (2014).

44. Trail, P. A. Antibody Directed Delivery for Treatment of Cancer: Antibody Drug Conjugates and Immunotoxins. Antibody-Drug Conjugates and Immunotoxins 3-22 (2013) .

45. Adams, G. P. & Weiner, L. M. Monoclonal antibody therapy of cancer. Nat. Biotechnol. 23, 1147-1157 (2005).

46. Kolmar, H. Natural and engineered cystine knot miniproteins for diagnostic and therapeutic applications. Curr. Pharm. Des. 17, 4329-4336 (2011).

47. Abbott, T. R. et al. Development of CRISPR as an Antiviral Strategy to Combat SARS-CoV-2 and Influenza. Cell 181, 865-876.e12 (2020).

48. Epel, M., Ellenhorn, J. D., Diamond, D. J. & Reiter, Y. A functional recombinant single-chain T cell receptor fragment capable of selectively targeting antigen-presenting cells. Cancer Immunol. Immunother. 51, 565-573 (2002).

1. A population of polypeptides comprising a sequence characterized as:
at least 85% sequence identity to SEQ ID NO:1, residues 1-124;
comprising a randomized region of from 1 to 10 amino acids within residues 12 and 22;
comprising two cysteine residues that form a disulfide bond;
wherein a polypeptide in the population binds to a Class II MHC protein with an affinity of less than 10⁶M.

2. A population of polypeptides comprising an amino acid sequence of SEQ ID NO:2, SEQ ID NO:3, or SEQ ID NO:4, wherein a polypeptide in the population binds to a Class II MHC protein with an affinity of less than 10⁻⁶M, optionally wherein the population comprises at least 10⁶ different sequences.

3. A population of polypeptides of claim 1, comprising the amino acid sequence of SEQ ID NO:4, optionally where the MHC Class II protein is HLA DR1.

4-5. (canceled)

6. A polypeptide selected from the population of claim 2, comprising an amino acid sequence of any of SEQ ID NO:2, SEQ ID NO:3, SEQ ID NO:4, or SEQ ID NO:5, wherein the polypeptide binds to an MHC Class II/peptide complex at an affinity of less than 10⁻⁶M, optionally wherein the peptide in the complex is a peptide of a pathogen antigen, a cancer-associated antigen, or an auto-antigen.

7. (canceled)

8. The polypeptide of claim 6, wherein the polypeptide is fused to an immune effector polypeptide, wherein the effector polypeptide is an Fc sequence, a chimeric antigen receptor, or a CD3-based bispecific T-cell engager.

9. (canceled)

10. A population of polypeptides according to claim 2, comprising a sequence as set forth in SEQ ID NO:5:

| 1 | 2 | 3 | 4 | 5 | 6 | 7 | 8 | 9 | 10 | 11 | 12 | 13 | 14 | 15 | 16 | 17 | 18 | 19 | 20 |
|---|---|---|---|---|---|---|---|---|---|---|---|---|---|---|---|---|---|---|---|
| M | K | L | R | C | E | N | P | K | K | A | X | X | X | N | X | Q | N | L | N |
| 21 | 22 | 23 | 24 | 25 | 26 | 27 | 28 | 29 | 30 | 31 | 32 | 33 | 34 | 35 | 36 | 37 | 38 | 39 | 40 |
| N | V | V | F | T | N | K | E | L | E | D | I | Y | D | L | S | N | K | E | E |
| 41 | 42 | 43 | 44 | 45 | 46 | 47 | 48 | 49 | 50 | 51 | 52 | 53 | 54 | 55 | 56 | 57 | 58 | 59 | 60 |
| T | K | E | V | L | K | L | F | K | L | K | V | N | Q | F | Y | R | H | A | F |
| 61 | 62 | 63 | 64 | 65 | 66 | 67 | 68 | 69 | 70 | 71 | 72 | 73 | 74 | 75 | 76 | 77 | 78 | 79 | 80 |
| G | I | V | N | D | Y | G | D | K | E | I | F | N | M | M | F | X | X | L | W |
| 81 | 82 | 83 | 84 | 85 | 86 | 87 | 88 | 89 | 90 | 91 | 92 | 93 | 94 | 95 | 96 | 97 | 98 | 99 | 100 |
| X | V | F | X | S | Q | X | X | X | A | N | N | V | E | X | I | K | X | N | I |
| 101 | 102 | 103 | 104 | 105 | 106 | 107 | 108 | 109 | 110 | 111 | 112 | 113 | 114 | 115 | 116 | 117 | 118 | 119 | 120 |
| X | X | L | D | W | I | M | A | E | A | D | N | D | L | C | Y | F | I | S | Q |

where X is any amino acid.

11. A population of polypeptides according to claim 10, comprising a sequence as set forth in SEQ ID NO:5, comprising one or more randomized residues as follows:
residue 9 is selected from K/D/V
residue 10 is selected from K/E/H
residue 12 is any amino acid other than C
residue 13 is any amino acid other than C
residue 14 is any amino acid other than C
residue 15 is selected from N/D/F/R
residue 16 is selected from A/L/M
residue 19 is selected from L/E/F/N/Q/W/Y
residue 22 is selected from V/L
residue 77 is selected from E/L/M/A/I/N/W
residue 78 is selected from L/M/F
residue 80 is selected from W/F/I/K/L/Y
residue 81 is selected from A/E/I/L/M/Q/S/T/V/W/Y
residue 82 is selected from D/I/L/M/Q/R/S/T/V/W
residue 85 is selected from S/I/L/M/T
residue 87 is selected from R/W/H/K/M/T
residue 88 is selected from F/Y/I/L/R/V/W
residue 89 is selected from N/S/A/F/W/Y
residue 92 is selected from D/N
residue 95 is selected from L/M/Q/V
residue 97 is selected from K/E
residue 98 is selected from F/A/L/T/W
residue 101 is selected from K/R/E/F
residue 102 is selected from M/F/I/L/T/V
residue 105 is selected from W/F/L/M
residue 108 is selected from A/K/R
residue 109 is selected from E/F/K/M/Q/V.

12. A population of polypeptides according to claim 10, comprising an amino acid sequence of SEQ ID NO:5 or SEQ ID NO:6, wherein a polypeptide in the population binds to a Class I MHC protein with an affinity of less than 10⁶M, optionally wherein the MHC Class I protein is an HLA-A*02 allele, optionally wherein the population comprises at least 10⁶ different sequences.

13-14. (canceled)

15. A polypeptide selected from the population of claim 12 comprising an amino acid of SEQ ID NO:6, wherein the polypeptide binds to an MHC Class I/peptide complex at an affinity of less than 10⁻⁶M, optionally wherein the peptide in the complex is a peptide of a pathogen antigen, a cancer-associated antigen, or an auto-antigen.

16. (canceled)

17. The polypeptide of claim 15, wherein the polypeptide is fused to an immune effector polypeptide, wherein the effector polypeptide is an Fc sequence, a chimeric antigen receptor, or a CD3-based bispecific T-cell engager.

18. (canceled)

19. A polypeptide comprising a sequence according to any of SEQ ID NO:10, 11, 12, 13 or 16.

20. A polypeptide selected from the population of claim 2, comprising a sequence according to SEQ ID NO:3, wherein residues 12-16 comprise a sequence selected from any of SEQ ID NO:17-SEQ ID NO:40.

21-33. (canceled)
